(19) United States
(12) Patent Application Publication
(10) Pub. No.: US 2026/0348933 A1
(43) Pub. Date: Nov. 5, 2026

(54) MONITORING SYSTEM AND APPARATUS FOR DETECTION OF HYDROGEN

(71) Applicant: COMMONWEALTH SCIENTIFIC AND INDUSTRIAL RESEARCH ORGANISATION, Acton, Australian Capital Territory (AU)

(72) Inventors: Jelena MARKOV, Acton, Australian Capital Territory (AU); Wen Shen MOW, Acton, Australian Capital Territory (AU); Mederic MAINSON, Acton, Australian Capital Territory (AU); Laurent LANGHI, Acton, Australian Capital Territory (AU); Emanuelle FRERY, Acton, Australian Capital Territory (AU); David GARDNER, Acton, Australian Capital Territory (AU)

(21) Appl. No.: 19/483,116

(22) PCT Filed: May 10, 2024
(86) PCT No.: PCT/AU2024/050462

§ 371 Date: Nov. 10, 2025

(30) Foreign Application Priority Data
May 10, 2023 (AU) 2023901421

(51) Int. Cl. G01N 33/00; G01N 33/24

(52) U.S. Cl. CPC G01N 33/005; G01N 33/0006; G01N 33/0034; G01N 33/0047; G01N 33/24

(57) ABSTRACT

Embodiments of the present invention is directed to a gas sensing apparatus configured to detect hydrogen (H₂) in the presence of one or more other gases. The gas sensing apparatus includes a first sensor for detecting at least hydrogen (H₂), a second sensor for detecting a gaseous species other than hydrogen (H₂), wherein the first sensor's response to the presence of hydrogen changes in the presence of the other gaseous species detected by the second sensor, and a wireless transmitter for wirelessly transmitting data from the apparatus to a remote station.

### CROSS-REFERENCE TO RELATED APPLICATIONS

This application is a National Stage Application of PCT/AU2024/050462, filed May 10, 2024, which claims benefit of priority to AU patent application No. 2023901421 filed on May 10, 2023, and which applications are incorporated herein by reference. To the extent appropriate, a claim of priority is made to each of the above disclosed applications.

### TECHNICAL FIELD

The present invention relates to a gas sensing apparatus, system and methods for detecting gas concentrations such as subsurface gas concentrations including but not limited to soil gas concentrations. In particular, embodiments of the present invention are directed to apparatus, systems and methods of detecting hydrogen (H₂) in the presence of one or more other gases in soil, although the scope of the invention is not necessarily limited thereto.

### BACKGROUND OF INVENTION

The detection of hydrogen in soil is an important aspect of soil analysis. It can provide valuable information for energy production, environmental monitoring, and agricultural production.

As the world transitions towards a clean energy and low emissions future, the importance of hydrogen as a key clean energy resource is increasingly being recognised. Hydrogen can provide a clean energy solution and a viable replacement for gas, petroleum and diesel fuels. The presence of hydrogen in the soil can indicate the potential for hydrogen fuel production through different geological processes.

Although hydrogen is an abundant element, it is highly reactive and significant accumulations of natural hydrogen have been considered rare. Moreover, challenges exist with the detection and exploration of natural hydrogen. Historically, samples from drillholes and deep mines have not been routinely sampled and analysed for hydrogen, so its presence is likely to be under-recorded. This is in part due to inappropriate analytical techniques and equipment. For example, some existing instrumentation for the detection of hydrogen are designed to take individual measurements at a single location at a time, and/or designed to take measurements from the surface of the soil and are therefore often impacted by bacterial activity in the ground.

In a recent study, hydrogen seeps were identified in the North Perth Basin. Seep characteristics can be determined by local geological and hydrological conditions, specifically whether hydrogen gas is seeping through soils and unconsolidated sediments, fractured bedrock or into water. However, current exploration equipment and techniques have been inadequate or ineffective, as they typically require invasive drilling. Moreover, in some instances, existing equipment may only be capable of providing instantaneous measurement of the soil-gas concentrations, or may require soft sediment for proper operation.

Furthermore, hydrogen is typically co-emitted with other gases such as methane (CH₄), helium (He), carbon dioxide (CO₂), nitrogen (N₂) and oxygen (O₂). The presence of these co-emitted gases can often affect the accuracy of the hydrogen detection when using conventional gas sensors. As hydrogen is often emitted with other gases, the conversions suggested by sensor manufacturers for determining the concentration of hydrogen based on raw sensor data (e.g. measurements of resistance) cannot be relied upon. Moreover, varying environmental conditions such as temperature, pressure and humidity can also impact the accuracy of hydrogen detection, potentially leading to inaccurate measurements of hydrogen concentrations using only conventional sensor technology.

In other fields such as environmental monitoring, hydrogen can be a key indicator of potential environmental issues. For example, high levels of hydrogen in the soil could suggest contamination by oil or gas leaks or other hazardous chemicals. Detecting these issues early can help prevent further contamination and minimize environmental damage.

In addition, hydrogen can also play a critical role in determining the soil's pH level, which can impact the soil's overall health. Measuring hydrogen levels can help determine if the soil is too acidic or too alkaline, which can then guide the necessary steps to adjust the pH level for healthy plant growth. Furthermore, hydrogen is a key element in the production of ammonia, which is used in fertilizers. Measuring hydrogen levels can provide valuable information for agricultural purposes, including understanding nutrient uptake and the efficacy of fertilizers.

Embodiments of the invention may provide an apparatus and system for remote sensing and monitoring of a target gaseous species such as hydrogen (H₂) in the presence of one or more other gases in soil, and a method of operation which overcomes or ameliorates one or more of the disadvantages or problems described above, or which at least provides the consumer with a useful choice.

A reference herein to a patent document or any other matter identified as prior art, is not to be taken as an admission that the document or other matter was known or that the information it contains was part of the common general knowledge as at the priority date of any of the claims.

### SUMMARY OF INVENTION

According to one aspect of the invention, there is provided a gas sensing apparatus configured to detect hydrogen (H₂) in the presence of one or more other gases, the apparatus including
a first sensor for detecting at least hydrogen (H₂),
a second sensor for detecting a gaseous species other than hydrogen (H₂), wherein the first sensor's response to the presence of hydrogen changes in the presence of the other gaseous species detected by the second sensor, and
a wireless transmitter for wirelessly transmitting data from the apparatus to a remote station.

Typically, the gas sensing apparatus is configured to detect hydrogen (H₂) in the presence of one or more other gases in subsurface environments.

In one embodiment, the gas sensing apparatus may be a soil gas sensing apparatus. The apparatus may be configured for long term placement in the soil.

In some applications, wells or boreholes may be provided in the ground for scientific or engineering purposes such as groundwater monitoring, mineral exploration, or geothermal energy extraction. In some embodiments, the gas sensing apparatus may be configured for placement in a well, a borehole or the like for the detection of one or more gases.

Advantageously, the gas sensing apparatus (e.g. soil gas sensing apparatus) according to embodiments of the present invention may be used to provide accurate detection of hydrogen quantities in the presence of other co-emitted gas species. Moreover, the apparatus may be placed in the soil long term to allow continuous monitoring of hydrogen concentrations in the soil over time. A system including a plurality of gas sensing apparatuses may be highly scalable to allow simultaneous and continuous monitoring of gas concentrations across an area of interest over time. In many applications, continuous subsurface/soil gas measurements of this nature may provide more useful information than instantaneous measurements. In particular, the apparatus may provide more accurate subsurface/soil gas measurements and capture changes in subsurface/soil gas concentration the longer the apparatus is deployed in the subsurface/soil environment when compared to instantaneous measurements. Furthermore, by placing the apparatus in the soil, the apparatus may be less likely to be impacted by above-surface conditions such as changes in local human activities, pollution, and the like.

The second sensor may be configured to detect any suitable co-emitted gas species. For example, the second sensor may detect any one or more of methane (CH₄), helium (He), carbon monoxide (CO), carbon dioxide (CO₂), ammonia (NH₃), and nitrogen dioxide (NO₂). In one embodiment, the second sensor may be configured to detect at least methane (CH₄). In another embodiment, the second sensor may be configured to detect at least helium (He). In a further embodiment, the second sensor may be configured to detect any one or more of carbon monoxide (CO), carbon dioxide (CO₂), ammonia (NH₃), and nitrogen dioxide (NO₂).

The gas sensing apparatus may be operatively configured to calibrate data from the first and second sensors to determine a concentration of hydrogen (H₂). In some embodiments, the gas sensing apparatus may be operatively configured to calibrate data from the first and second sensors to determine a concentration of any target gas species.

The apparatus may be operatively configured to calibrate data from the first and second sensors to determine a concentration of any one or more of methane (CH₄), helium (He), carbon monoxide (CO), carbon dioxide (CO₂), ammonia (NH₃), and nitrogen dioxide (NO₂).

The second sensor may be configured to detect at least methane (CH₄). The apparatus may further include a third sensor configured to detect any one or more of carbon monoxide (CO), carbon dioxide (CO₂), ammonia (NH₃), and nitrogen dioxide (NO₂), wherein the first sensor's response to the presence of hydrogen changes in the presence of any one or more of methane (CH₄), carbon monoxide (CO), carbon dioxide (CO₂), ammonia (NH₃), and nitrogen dioxide (NO₂).

In some embodiments, the second sensor may be configured to detect at least methane (CH₄), and the apparatus may further include a third sensor configured to detect at least helium (He). Typically, the first sensor's response to the presence of hydrogen changes in the presence of any one or both of methane (CH₄) and helium (He).

In some embodiments, the apparatus may include a microprocessor operatively configured to calibrate sensor data collected by the sensors on board to determine a gas concentration of one or more gaseous species detected by the apparatus. In these embodiments, data transmitted by the wireless transmitter to the remote station may include calibrated data presenting concentration values of the detected gaseous species. In some embodiments, the data transmitted by the wireless transmitter to the remote station may include uncalibrated raw sensor data, and calibration of the raw sensor data may be carried out by the remote station, or a processor associated with the remote station.

The apparatus may be operatively configured to calibrate data from the first, second and third sensors to determine a concentration of at least one of methane (CH₄), carbon monoxide (CO), carbon dioxide (CO₂), ammonia (NH₃), and nitrogen dioxide (NO₂).

The gas sensing apparatus may be operatively configured to calibrate data from the first, second and third sensors to determine a concentration of at least one of methane (CH₄) and helium (He).

The gas sensing apparatus may be operatively configured to calibrate data from the first, second and third sensors to determine a concentration of hydrogen (H₂).

Any one or more sensors may be integrated into a single sensor unit, or separately provided sensor units. For example, a single sensor unit may include one sensor for detecting a single gaseous species (e.g. H₂), or a plurality of sensors to detect a plurality of gaseous species (e.g. H₂ and CH₄). In other words, any one or more of the sensors (e.g. first, second and third sensors) may be provided as separate sensor modules or integrated in a single sensor module assembly. In some embodiments, a single sensor module may include sensing elements for detecting one or more different gaseous species simultaneously.

In some embodiments, the gas sensing apparatus may further include an environmental sensor configured to detect any one or more of temperature, humidity and pressure.

The apparatus may be operatively configured to perform the calibration based on one or more statistical and/or machine learning models. In particular, the apparatus may be operatively configured to perform the calibration via a supervised machine learning model. The supervised machine learning model may be pre-trained based on sensor data indicative of concentrations of hydrogen (H₂) and one or more gaseous species other than hydrogen (H₂), and actual gas concentrations of hydrogen (H₂) and one or more gaseous species other than hydrogen (H₂) corresponding to the sensor data.

The supervised machine learning model may include any one of a linear regression model, gradient boosting machine learning, or random forest algorithm.

In some embodiments, the gas sensing apparatus may further include a battery module. In some embodiments, the gas sensing apparatus may be powered by an external power source. For example, the apparatus may be solar powered.

The apparatus may include a housing for containing the sensors therein. The housing may have an elongate body, and the sensors may be mounted proximate a first end of the elongate body. In use, the elongate body of the apparatus may be inserted into soil for long term continuous monitoring of gas concentrations.

The gas sensing apparatus may further include a barrier disposed at the first end of the body. The barrier may serve to protect componentry (e.g. electronics) within the housing of the apparatus, for example by preventing moisture and contaminants from entering the housing of the apparatus. Any suitable barrier may be used. In some embodiments, the barrier may include a gas-permeable membrane. In particular, the membrane may include pores having a pore size of about 1 μm to 10 μm. Moreover, the membrane may have a thickness of roughly 100 μm to 300 μm. The membrane may be configured to more readily permit passage of certain gaseous species (such as hydrogen), whilst filtering out or inhibiting passage of other gaseous species. The membrane may be made from any suitable material. In one embodiment, the membrane is made from Polytetrafluoroethylene (PTFE). The membrane may be hydrophilic or hydrophobic, laminated or unlaminated.

Within the elongate body of the apparatus, the sensors may be spaced from the barrier so as to define a cavity therebetween. Typically, the membrane allows an equilibrium of gases between the cavity and the gases immediately outside the first end of the body. In some embodiments, the apparatus may include a pump or fan to periodically extract gases from the housing or cavity so as to enable measurement of gas flux by the apparatus. In some embodiments, depending on the specific arrangement of sensors in the apparatus, the apparatus may be configured to flush gases from the cavity or internal spaces of the housing, which may include a cavity associated with the sensor modules.

In one embodiment, the apparatus may include an inflow conduit for providing a flow of gases into the cavity via the inflow conduit. The apparatus may further include an outflow conduit for permitting a flow of gases to exit the cavity via the outflow conduit. The inflow conduit may provide fluid communication between the cavity and atmospheric gases externally of the apparatus housing. The outflow conduit may provide fluid communication between the cavity and atmospheric gases externally of the apparatus housing.

The apparatus may include an inflow valve coupled to the inflow conduit. The apparatus may further include an outflow valve coupled to the outflow conduit. The inflow valve and/or the outflow valve may be solenoid valves. Moreover, the inflow valve may be a three-way valve.

The apparatus may further include a reference gas source coupled to the inflow valve and inflow conduit.

A method of flushing the cavity to facilitate determination of gas flux may include flushing the cavity with one or more reference gases. In one embodiment, the reference gases may be provided by the reference gas source. In one embodiment, the reference gases may be drawn from atmospheric gases.

More specifically, the method of flushing the cavity to facilitate determination of gas flux may include operating the pump to move gases into the cavity, determining a concentration of a target gaseous species, comparing the concentration of the target gaseous species with a threshold value and disabling the pump once the concentration of the target gaseous species is below the threshold value. The method may further include sampling sensor data to determine gas flux based on a change of concentration of the target gaseous species over time.

To enable wireless communication between the apparatus and the remote station, the gas sensing apparatus may further include an antenna for wirelessly transmitting data from the apparatus to the remote station. In some embodiments, the apparatus may further include at least one data port to enable transmission of data from the apparatus via a wired connection. In some embodiments, the gas sensing apparatus may be configured for connection with the internet.

The apparatus may form part of a network of apparatuses. In practice, a plurality of apparatuses may be distributed over an area of interest to monitor hydrogen concentrations continuously (e.g. for the exploration of natural hydrogen), and other gas species of interest over any suitable period of time. Each apparatus within the network may be placed in the soil to collect gas concentration data and configured to transmit the collected data to the remote station or upload the information to a cloud server, to thereby allow remote and autonomous monitoring of gas concentrations, and in particular hydrogen concentrations, over the entire area of interest over a period of time.

According to yet another aspect of the invention, there is provided a gas sensing apparatus configured to detect a first gaseous species in the presence of one or more other gaseous species, the apparatus including
a first sensor for detecting at least a first gaseous species,
a second sensor for detecting at least a second gaseous species, wherein the first gaseous species is different to the second gaseous species, and wherein the first sensor's response to the presence of the first gaseous species changes in the presence of the second gaseous species detected by the second sensor, and
a wireless transmitter for wirelessly transmitting data from the apparatus to a remote station.

According to another aspect of the invention, there is provided a sensing apparatus configured to detect hydrogen (H₂) in the presence of varying environmental conditions, the apparatus including
a first sensor for detecting at least hydrogen (H₂),
a second sensor for detecting an atmospheric condition including any one or more of temperature, pressure and humidity, wherein the first sensor's response to the presence of hydrogen changes with varying environmental conditions as detected by the second sensor, and
a wireless transmitter for wirelessly transmitting data from the apparatus to a remote station.

According to yet another aspect of the invention, there is provided a sensing apparatus configured to detect a gas species in the presence of varying environmental conditions, the apparatus including
a first sensor for detecting the gas species, a second sensor for detecting an environmental condition including any one or more of temperature, pressure and humidity, wherein the first sensor's response to the presence of detected gas species changes with varying environmental conditions as detected by the second sensor, and
a wireless transmitter for wirelessly transmitting data from the apparatus to a remote station.

Whilst many of the example embodiments described herein relate to the detection of hydrogen (H₂), the inventive concept may be similarly applied to the detection of any gaseous species in the presence of one or more other gaseous species and/or varying environmental conditions, as described in further detail herein.

According to another aspect of the invention, there is provided a system including one or more gas sensing apparatuses as described herein.

The system may further include the remote station for receiving data from the one or more gas sensing apparatuses, wherein the remote station is operatively configured to calibrate data received from each apparatus to determine a concentration of hydrogen (H₂).

As previously mentioned, the remote station may receive raw sensor data and/or pre-processed sensor data from each of the gas sensing apparatuses. The remote station may be operatively configured to calibrate data received from each apparatus to determine a concentration of at least one of hydrogen (H₂), methane (CH₄), carbon monoxide (CO), carbon dioxide (CO₂), ammonia (NH₃), and nitrogen dioxide (NO₂).

The remote station may be operatively configured to perform the calibration based on one or more statistical and/or machine learning models. The remote station may be operatively configured to perform the calibration via a supervised machine learning model, the supervised machine learning model being pre-trained based on
sensor data indicative of concentrations of hydrogen (H₂) and one or more gaseous species other than hydrogen (H₂), and
actual gas concentrations of hydrogen (H₂) and one or more gaseous species other than hydrogen (H₂) corresponding to the sensor data.

The supervised machine learning model may include any one of a linear regression model, gradient boosting machine learning, or random forest algorithm.

The system may further include a graphical user interface for providing a visual display of calibrated gas concentration data. The system may include a processor for generating display data for the graphical user interface to provide a graphical representation of the calibrated gas concentration data in real time or near real time.

Any suitable wireless communications protocols may be used to enable wireless communication between each apparatus and the remote station. Typically, the wireless communication between each apparatus and the remote station may be bidirectional wireless communication. For example, the wireless communication between the one or more gas sensing apparatuses and the remote station may be enabled via LoRa Wide Area Network (LoRaWAN). In other embodiments, wireless communication between the one or more gas sensing apparatuses and the remote station may be enabled via low-power, low-data-rate wireless communication protocols such as Zigbee. In a further embodiment, wireless communication between the one or more gas sensing apparatuses and the remote station may be enabled via a wireless mesh networking protocol such as DigiMesh. In a DigiMesh network, each apparatus may act as a router and forward data to other apparatuses in the network. This may create a self-healing mesh topology that can provide greater range and reliability than point-to-point wireless communication. DigiMesh may also be used to support multi-hop routing, which allows data to be transmitted over longer distances by passing through multiple apparatuses.

In some embodiments, each apparatus may be configured for direct cloud connectivity, optionally bypassing the remote station in some instances. To enable direct cloud connectivity, each apparatus may be enabled with Long-Term Evolution (LTE) wireless communication, or 4G LTE. Alternatively, each apparatus may be configured for direct internet connectivity via Internet of Things (IoT), or more specifically, a low-power, wide area network (LPWAN) wireless communication network such as Narrowband Internet of Things (NB-IoT).

The system may further include one or more solar modules for powering the one or more gas sensing apparatuses.

According to a further aspect of the invention, there is provided a method of determining a concentration of hydrogen (H₂) in the presence of one or more other gases, the method including 1sensing, via first sensor, hydrogen (H₂),
sensing, via a second sensor, a gaseous species other than hydrogen (H₂), wherein the first sensor's response to the presence of hydrogen (H₂) changes in the presence of the other gaseous species detected by the second sensor, and
calibrating sensor data from the first and second sensors to determine a concentration of hydrogen (H₂).

The step of sensing via the second sensor may include sensing at least methane (CH₄), and the method may further include
sensing any one or more of helium (He), carbon monoxide (CO), carbon dioxide (CO₂), ammonia (NH₃), and nitrogen dioxide (NO₂).

The method may further include calibrating sensor data to determine a concentration of any one or more of methane (CH₄), helium (He), carbon monoxide (CO), carbon dioxide (CO₂), ammonia (NH₃), and nitrogen dioxide (NO₂).

Calibrating sensor data may include calibrating sensor data based on one or more statistical and/or machine learning models. Calibrating sensor data may include calibrating sensor data based on a supervised machine learning model, wherein the supervised machine learning model is pre-trained based on
sensor data indicative of concentrations of hydrogen (H₂) and one or more gaseous species other than hydrogen (H₂), and
actual gas concentrations of hydrogen (H₂) and one or more gaseous species other than hydrogen (H₂) corresponding to the sensor data.

In some embodiments, the method may be a method of determining a concentration of hydrogen (H₂) in the presence of one or more other gases in soil. The sensing may include sensing, via first sensor, hydrogen (H₂) in the soil, and sensing, via a second sensor, a gaseous species other than hydrogen (H₂) in the soil.

It will be appreciated that the apparatus does not necessarily have to be inserted into and/or be in contact with soil to detect gases in soil (soil gases). In some embodiments, the apparatus may be deployed in well or boreholes, tunnels, basements or any other suitable underground or excavated structures. In some embodiments, the apparatus may be deployed above ground. For example, the apparatus may be deployed on or near a surface of the ground.

According to a yet another aspect of the invention, there is provided a computer-implemented method of determining a concentration of hydrogen (H₂) in the presence of one or more other gases in soil, the method including
receiving sensor data representing a sensed concentration of hydrogen (H₂) in the soil and a sensed concentration of a gaseous species other than hydrogen (H₂), wherein the sensed concentration of hydrogen (H₂) is influenced by the sensed concentration of the other gaseous species, and
calibrating the sensor data to determine a concentration of hydrogen (H₂).

The computer-implemented method of claim 39, wherein the sensor data represents a sensed concentration of hydrogen (H₂) in the soil and a sensed concentration of any one or more of methane (CH₄), helium (He), carbon monoxide (CO), carbon dioxide (CO₂), ammonia (NH₃), and nitrogen dioxide (NO₂).

The computer-implemented method may further include calibrating the sensor data to determine a concentration of any one or more of methane (CH₄), helium (He), carbon monoxide (CO), carbon dioxide (CO₂), ammonia (NH₃), and nitrogen dioxide (NO₂).

In the computer-implemented method, calibrating the sensor data may include calibrating the sensor data based on one or more statistical and/or machine learning models. In particular, calibrating sensor data may include calibrating sensor data based on a supervised machine learning model, wherein the supervised machine learning model is pre-trained based on
sensor data indicative of concentrations of hydrogen (H₂) and one or more gaseous species other than hydrogen (H₂), and
actual gas concentrations of hydrogen (H₂) and one or more gaseous species other than hydrogen (H₂) corresponding to the sensor data.

In some embodiments of the computer-implemented method, the supervised machine learning model may include any one of a linear regression model, gradient boosting machine learning, or random forest algorithm.

According to a further aspect of the invention, there is provided a non-transitory computer readable medium having stored thereon software instructions that when executed by a processor, causes the processor to perform the computer implemented method as described herein.

According to one aspect of the invention, there is provided a gas sensing apparatus configured to detect hydrogen (H₂) in the presence of one or more other gases in soil, the apparatus being configured for long term placement in the soil, the apparatus including
a first sensor for detecting at least hydrogen (H₂),
a second sensor for detecting a gaseous species other than hydrogen (H₂), wherein the first sensor's response to the presence of hydrogen changes in the presence of the other gaseous species detected by the second sensor, and
a wireless transmitter for wirelessly transmitting data from the apparatus.

In one embodiment, the apparatus may be configured for internet connection via the wireless transmitter. In one embodiment, the apparatus may be configured for wireless communication with a remote station via the wireless transmitter.

In order that the invention may be more readily understood and put into practice, one or more preferred embodiments thereof will now be described, by way of example only, with reference to the accompanying drawings.

It will be appreciated that for simplicity and clarity of illustration, where considered appropriate, reference numerals may be repeated among the figures to indicate corresponding or analogous elements.

### BRIEF DESCRIPTION OF DRAWINGS

FIG. 1A is a schematic diagram illustrating a gas sensing apparatus inserted into soil during deployment in accordance with one embodiment of the present invention.

FIG. 1B is a schematic diagram illustrating the gas sensing apparatus inserted into a well or borehole during deployment in accordance with one embodiment of the present invention.

FIG. 2A is an end view of the gas sensing apparatus as shown in FIGS. 1A and 1B.

FIG. 2B is the A-A cross sectional view of the gas sensing apparatus in accordance with FIG. 2A.

FIG. 2C is the C-C cross sectional view of the gas sensing apparatus in accordance with FIG. 2B.

FIG. 2D is an enlarged view of a first end portion B of the gas sensing apparatus in accordance with FIG. 2C.

FIG. 2E is a mounting portion of the gas sensing apparatus as shown in FIG. 2B.

FIG. 3A illustrates a schematic circuit diagram of a circuit module of the gas sensing apparatus as shown in FIGS. 2A to 2E according to one embodiment.

FIG. 3B illustrates a schematic circuit diagram of a circuit module of the gas sensing apparatus as shown in FIGS. 2A to 2D according to another embodiment.

FIG. 3C illustrates a schematic diagram of a gas sensing apparatus including a pump assembly coupled to the circuit module shown in FIG. 3B according to another embodiment.

FIG. 3D is a process flow chart illustrating a method of flushing the apparatus of FIG. 3C to determine gas flux.

FIG. 4A illustrates the placement of a network of gas sensing apparatuses over an area of interest in a system for remote monitoring of hydrogen concentrations in soil according to embodiments of the invention.

FIG. 4B illustrates altered placement of a network of gas sensing apparatuses over an area of interest in a system for remote monitoring of hydrogen concentrations in soil, the altered placement being based on measured gas concentration data obtained from deployment according to FIG. 4A.

FIG. 4C illustrates further altered placement of a network of gas sensing apparatuses over an area of interest in a system for remote monitoring of hydrogen concentrations in soil, the further altered placement being based on measured gas concentration data obtained from deployment according to FIG. 4B.

FIG. 5 is a graph illustrating data associated with a predictive model used to calibrate sensor data using linear regression in a method of determining a concentration of hydrogen according to an embodiment of the present invention.

FIG. 6A is a graph illustrating gas concentration data recorded in an experiment to generate training data for a machine learning model in a method of determining a concentration of hydrogen according to an embodiment of the present invention.

FIG. 6B is a graph illustrating raw sensor data recorded in an experiment to generate training data for a machine learning model in a method of determining a concentration of hydrogen according to an embodiment of the present invention.

FIG. 7 is a scatter plot illustrating the accuracy of calibration using gradient boosting machine learning to calibrate sensor data to determine concentrations of hydrogen.

FIG. 8 is a scatter plot illustrating the accuracy of calibration using a random forest regression model to calibration sensor data to determine concentrations of hydrogen.

FIG. 9A is a graph illustrating target concentrations of hydrogen in an experimental set up to obtain training data for the statistical or machine learning model.

FIGS. 9B to 9M illustrate sensor responses of the gas sensing apparatus during the experiment of FIG. 9A.

FIG. 10A is a graph illustrating target concentrations of hydrogen, methane, and helium in an experimental set up to obtain training data for the statistical or machine learning model.

FIGS. 10B to 10M illustrate sensor responses of the gas sensing apparatus during the experiment of FIG. 10A.

FIGS. 11A and 11B are graphs illustrating target concentrations of hydrogen and methane in an experimental set up to obtain training data for the statistical or machine learning model.

FIGS. 11C to 11N illustrate sensor responses of the gas sensing apparatus during the experiment of FIGS. 11A and 11B.

FIGS. 12A to 12L illustrate sensor responses of the gas sensing apparatus during a further experiment to obtain training data for the statistical or machine learning model in an outdoor setting.

FIG. 13 illustrates a correlation matrix of the variables used to train a machine learning model.

FIG. 14 illustrates a graphical user interface associated with the remote station according to one embodiment of the invention.

### DETAILED DESCRIPTION

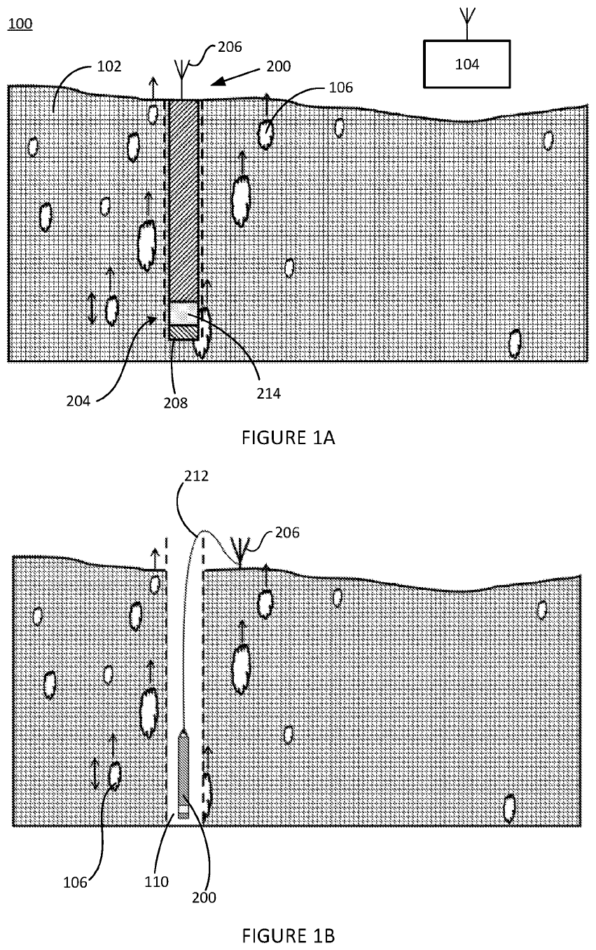

FIG. 1A illustrates a gas monitoring environment 100 for monitoring gas seeps such as hydrogen and methane seeps from soil 102. In this environment 100, gases 106 gradually migrate from underground towards the surface of the soil 102. A gas sensing apparatus 200 configured to detect at least hydrogen (H₂) may be inserted into the ground for long term placement in the soil 102. The apparatus 200 may be configured to detect hydrogen in the presence of one or more other gases in the soil 102. Typically, hydrogen is co-emitted with other gaseous species, including methane (CH₄), helium (He), carbon dioxide (CO₂), nitrogen (N₂) and oxygen (O₂), with methane being the most commonly occurring co-emitted gas.

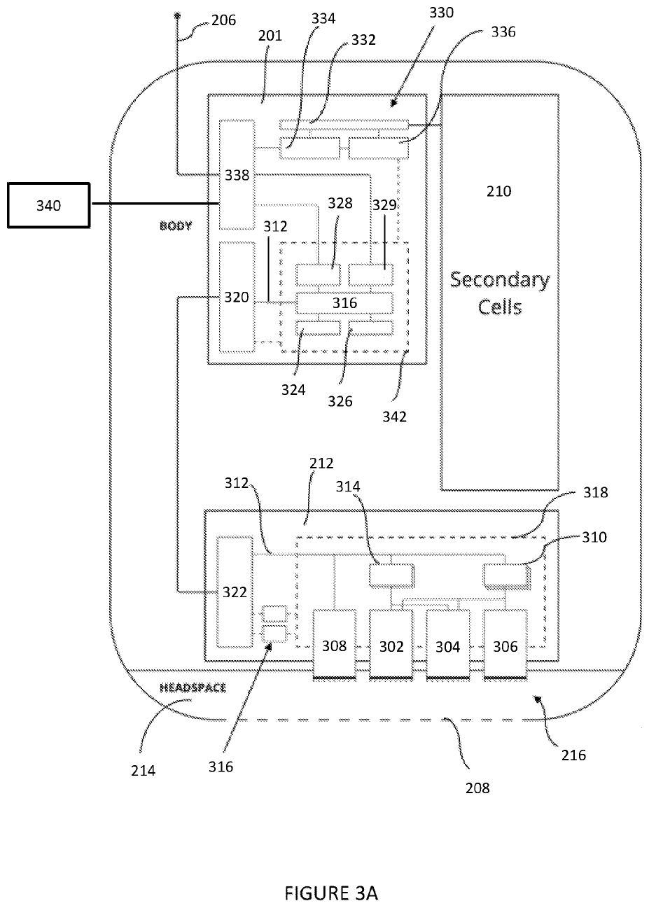

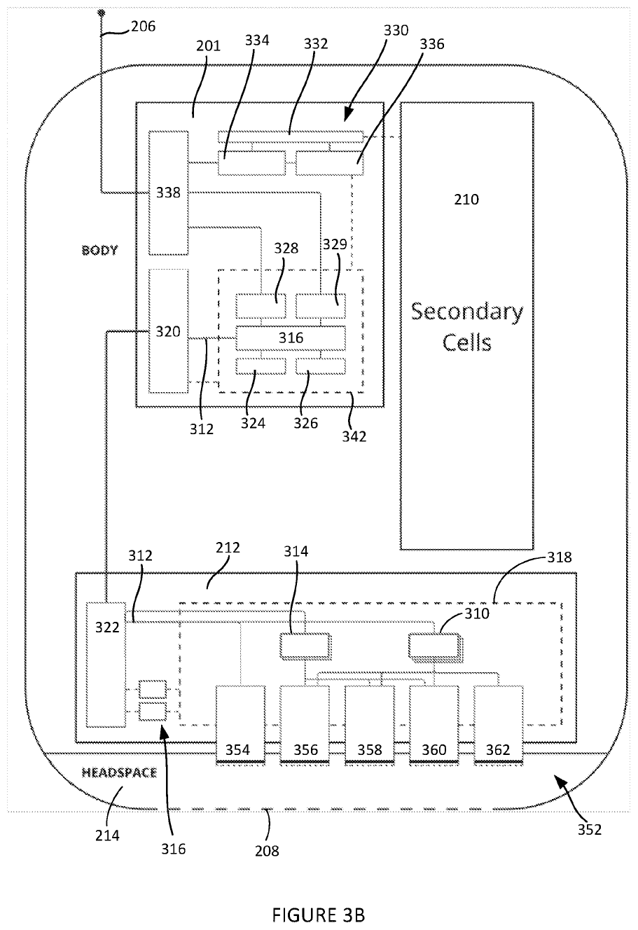

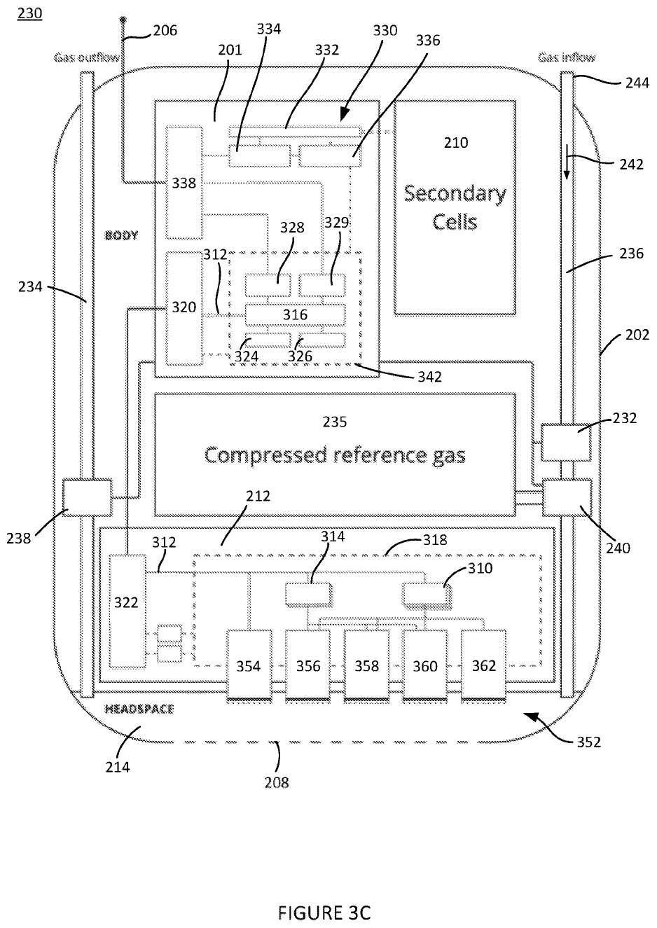

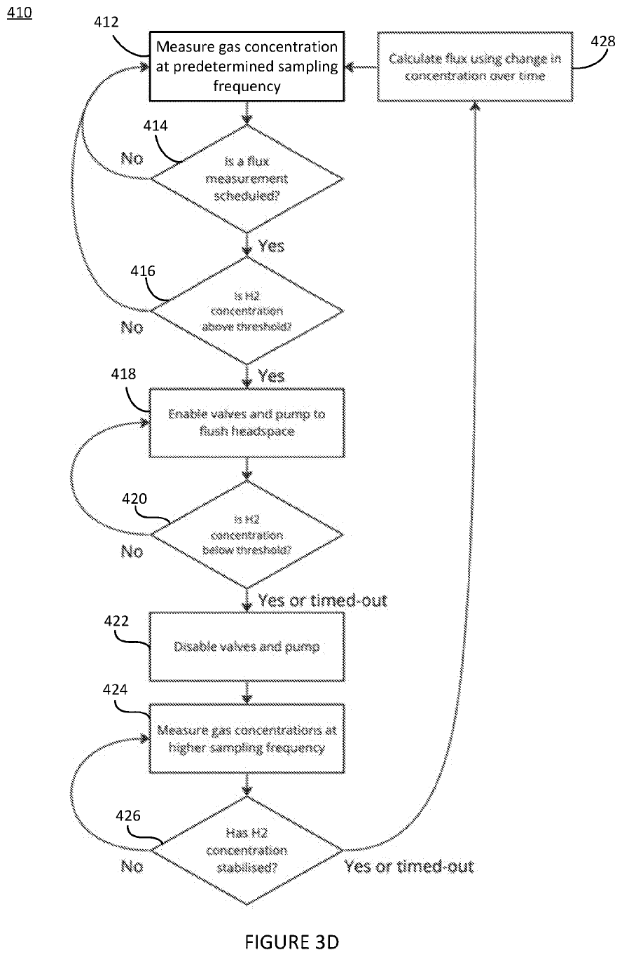

The apparatus 200 includes a first sensor 302 for detecting at least hydrogen (H₂), and one or more other sensors 304, 306 for detecting a co-emitted gaseous species other than hydrogen (H₂), such as methane (CH₄), helium (He), carbon monoxide (CO), carbon dioxide (CO₂), ammonia (NH₃), or nitrogen dioxide (NO₂) (see FIG. 3A). The presence of these co-emitted gases can affect the accuracy of each sensor 302, 304, 306 for determining the concentration of each individual gaseous species of interest, such as hydrogen. In other words, each sensor's response to the presence of a particular gaseous species of interest (e.g. hydrogen) changes in the presence of the other gaseous species. The other gaseous species may be detected by the one or more other sensors 304, 306 in the apparatus 200, 230. As discussed in further detail below, whilst example embodiments described herein relate to the detection of hydrogen (H₂), the gas sensing apparatus may be configured to detect any gaseous species of interest, for example methane (CH₄), helium (He), carbon monoxide (CO), carbon dioxide (CO₂), ammonia (NH₃), or nitrogen dioxide (NO₂).

The apparatus 200 also includes an antenna 206 coupled to a wireless transmitter 328 for wirelessly transmitting data (e.g. sensor data) from the apparatus 200 to a remote station 104. The remote station 104 may be located at any suitable location with respect to the apparatus 200, or a network of a plurality of apparatuses as described in further detail below.

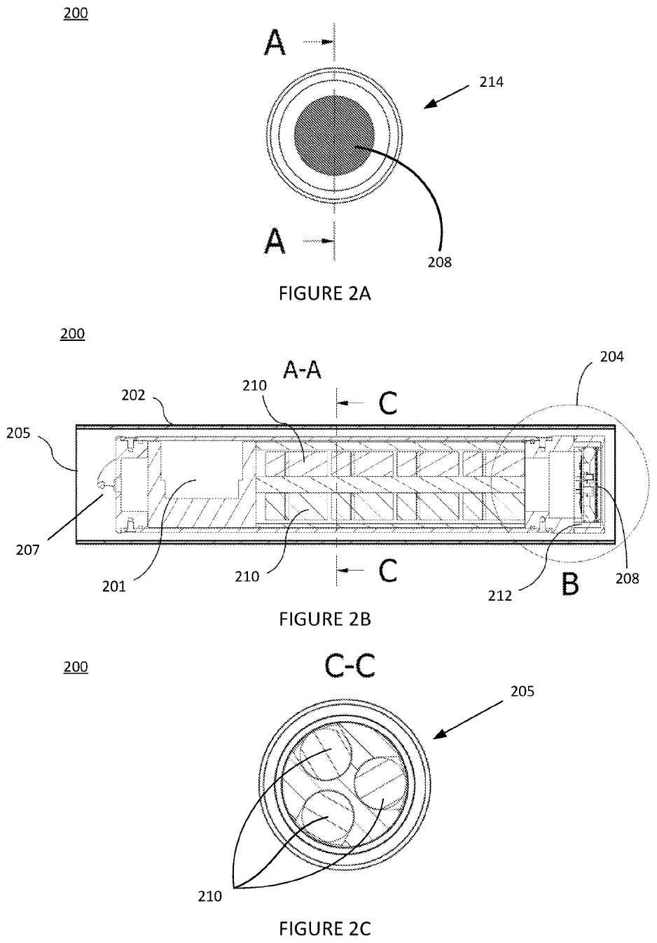

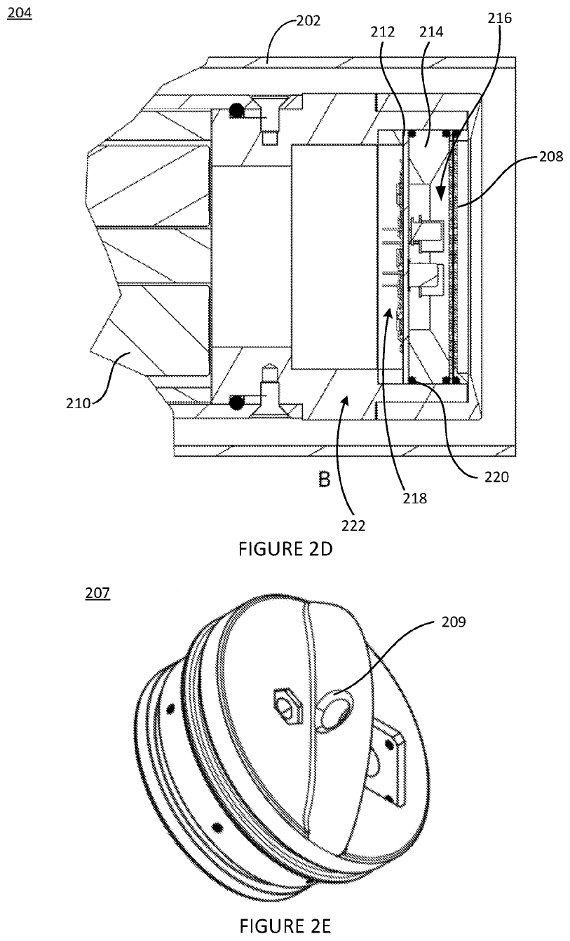

In the embodiment shown, the apparatus 200 has a housing 202 having a generally cylindrical elongate body. However, in other embodiments, the housing 202 may have any other suitable regular or irregular shape. The elongate body of the housing 202 has a first end 204 portion forming a base of the apparatus 200 in use, when the apparatus 200 is inserted into the soil 102. As explained in further detail below with reference to FIGS. 2B, 2D and 3A to 3C, a sensor circuit 212 is mounted internally of the housing 202 proximate the first end 204 of the elongate body and spaced from a barrier 208. As shown in FIG. 1A, the apparatus 200 is inserted into the soil 102 with its first end 204 facing downwardly such that the barrier 208 is facing downwardly and exposed to the soil and soil gases in use. As gas pockets 106 gradually move from deep underground towards the surface of the soil 102, a mixture of gases penetrate the barrier 208 and enter the apparatus 200. In particular, the barrier 208 allows the gases to pass into a cavity 214 between the sensor circuit 212 and the barrier 208 to facilitate detection of gases by the sensors 216. This will be explained in further detail below with reference to FIGS. 2B, 2D and 3A. The barrier 208 protects the apparatus 200 by preventing moisture and other contaminants from entering the housing 202 and cavity 214 to thereby prevent damage of electronic components which may lead to malfunctioning of the apparatus 200.

As it will be appreciated, it is not necessary for the gas sensing apparatus 200 to be inserted into soil to detect soil gases. As illustrated in FIG. 1B, the gas sensing apparatus 200 may be deployed in a well or borehole 110 to detect soil gases 106. In this scenario, soil gases 106 may travel into the well or borehole 110 for detection by the gas sensing apparatus 200. Similarly, the gas sensing apparatus 200 may be deployed in a basement or tunnel or any other suitable underground or excavated structure.

In the particular embodiment shown in FIG. 1B, the apparatus 200 including a mounting mechanism 207 to facilitate securing of the apparatus 200 to an anchor (e.g. antenna 206) by a tether 112 so that the apparatus 200 can be positioned at any desirable depth within the well or borehole 110.

An end view of the apparatus 200 illustrating the first end 204 is shown in FIG. 2A. As mentioned, the barrier 208 is exposed at the first end 204 to permit gases 106 from the soil 102 to enter the cavity 214 for detection by the sensors 216, whilst protecting the apparatus 200 by preventing moisture and contaminants from entering the housing 202.

Now referring to FIG. 2B, which illustrates the A-A cross sectional view of the apparatus 200 taken across the length of its elongate body. Within the housing 202 of the apparatus 200, space is provided for a battery assembly having a plurality of battery units 210. Layers of battery units 210 are stacked lengthwise along the elongate body of the apparatus 200. As shown in FIG. 2C (C-C cross sectional view of FIG. 2B), each layer includes three battery units 210. In the particular embodiment shown in FIGS. 2B and 2C, space for four layers of battery units 210 are provided, giving the apparatus 200 capacity to hold 12 battery units. In other embodiments, the apparatus 200 may be configured to hold any suitable number of battery units internally and/or externally of the housing 202.

In some embodiments, the apparatus 200 may further include mounting portion 207 proximate a second end 205 of the apparatus 200. The mounting portion 207 is more clearly shown in FIG. 2E, and may form a removable and/or sealing cap for the second end 205 of the apparatus. In particular, the mounting portion 207 may include an aperture for receiving a tether for tethering the apparatus 200 to an anchor, for example when the apparatus 200 is deployed in a well, borehole 110 or the like (for example, see FIG. 1B). In other embodiments, any suitable mounting mechanism 207 may be used to enable tethering of the apparatus 200 to an anchor. For example, the mounting portion 207 may include a hook, a clamp, clip or any other suitable fastening mechanism to facilitate attachment of the apparatus 200 an anchor to thereby support appropriate placement of the apparatus 200 at any suitable location within the well or borehole 110.

At the first end portion 204, the apparatus 200 provides the barrier 208 and sensor circuit 212 spaced from the barrier 208. A controller circuit 201 is also provided in the housing 202 proximate the second end 205 of the apparatus 200. As explained in further detail below with reference to FIG. 3A, the control circuit 201 receives data from the sensor circuit 212 and transmits the data wirelessly to the remote station 104. Providing a control circuit 201 printed circuit board (PCB) that is separate to the sensor circuit 212 PCB in the apparatus 200 in this manner provides a number of advantages. For example, providing two separate PCBs allows the distance of digitisers and drivers to sensors 216 to be minimised in the sensor circuit 212 so as to achieve better noise immunity for the sensor signals. In addition, the size of the sensor circuit 212 can be minimised thereby allowing reduction in size for the first end portion 204 of the apparatus 200 so as to achieve an improved gas exchange response. Moreover, having a separate control circuit 201 provides better freedom of design for the control circuit 201 in terms of its physical footprint, digital interference with the sensors 216, and application-dependant communication implementation. Furthermore, providing two separate PCBS allows decoupling of thermal effects of the sensor circuit 212 PCB from the control circuit 201 PCB. For instance, any electronic components having poor-temperature tolerance may be located on the control circuit 201 so as to be separated from the temperature sensitive components of the sensor circuit 212.

An enlarged view of the first end portion 204 of the apparatus 200 is shown in FIG. 2D. The barrier 208 covers an opening in the housing 202 at the first end 204 of the apparatus 200. Accordingly, the barrier 208 is exposed through the housing 202. In the embodiment shown in FIG. 2D, the barrier is a hydrophobic gas-permeable membrane 208. In particular, the membrane 208 may include pores having a pore size of about 1 μm to 5 μm. The membrane 208 may have a thickness of about 100 μm to 300 μm.

A sensor circuit 212 having gas sensors 216 for the detection of hydrogen and other co-emitted gaseous species is also provided in the first end portion 204 of the apparatus 200. The sensor circuit 212 is spaced from the membrane 208 so as to define a cavity 214 therebetween. In use, the membrane 208 permits gases 106 from the soil 102 to enter the apparatus housing 202 and equilibrate within the cavity 214 to allow detection of the gaseous species by the sensors 216. As mentioned, the barrier serves to protect internal components of the apparatus 200 by preventing moisture and other contaminants from entering the housing 202.

The sensor circuit 212 is configured such that the sensors 216 are disposed on one side of the circuit 212 facing the membrane 208, and remaining circuit electronics 218 are disposed on an opposite side of the circuit 212. A sealed cavity 214 is provided, for example via use of an O-ring 220 so as to avoid any contamination and/or moisture from entering the remainder of the apparatus housing 222. Contamination and/or moisture within the remainder of the housing 222 may interfere with the proper operation of the battery units 210 and circuit electronics 218 and/or cause damage over time.

The configuration of the apparatus circuit module 300 according to one example embodiment will now be described in further detail with reference to FIG. 3A. The circuit module 300 includes sensor circuit 212 (mounted proximate the first end 204 of the apparatus 200), and a control circuit 201 (typically spaced from the first end 204 and may be mounted proximate a second end 205 of the apparatus 200 opposite the first end 204). As previously mentioned, separating the control circuit 201 printed circuit board (PCB) from the sensor circuit 212 PCB as illustrated in FIG. 2B may advantageously allow for better freedom of circuitry design in terms of maximising the signal-to-noise ratio of sensor data, optimising the gas exchange response, reducing physical footprint, and thermal effects.

The sensor circuit 212 includes four sensor modules 216, including a chemiresistive hydrogen (H₂) gas sensor 302 (e.g. the first sensor) for detecting a concentration of hydrogen, a chemiresistive methane (CH₄) gas sensor 304 (e.g. the second sensor) for detecting a concentration of methane, a thermoconductive gas sensor 306 (e.g. the third sensor) for detecting concentrations of a plurality of gaseous species including helium (He), carbon monoxide (CO), ammonia (NH₃), and nitrogen dioxide (NO₂), and an atmospheric sensor 308 for detecting temperature, humidity and pressure.

In the embodiment shown in FIG. 3A, the atmospheric sensor 308 is a digital sensor, and gas sensors 302, 304, 306 are analogue sensors. Digital sensor data from the atmospheric sensor 308 is transmitted to microprocessor 316 via 12C communications link 312. Analogue sensor data from gas sensors 302, 304, 306 is converted to digital data via analogue to digital converter 310 before transmission to the microprocessor 316 via the 12C communications link 302. In alternative embodiments, any one or more of the sensors 216 may be digital or analogue.

Current controlled heater drivers 314 may be provided to facilitate regulating power delivered to chemiresistive sensors 302, 304 so that variable power may be delivered to the sensors 302, 304 based on a voltage drop measured for each of the sensors 302, 304 so as to enable optimum sensor performance, and to avoid overheating. Power adapters 316 may also be provided to modulate current delivered to the sensor circuit 202. The power flow region 318 of the sensor circuit 212 is illustrated using dashed lines. The sensor circuit 212 is coupled to the controller circuit 201 via connectors 320, 322.

The controller circuit 201 includes microprocessor 316 configured to receive sensor data from the sensors 216 via communications link 312. In some embodiments, a memory device 324, such as a microSD card may be coupled to the microprocessor 316 for storing/backing up sensor data and/or calibration data. A battery-backed real time clock device 326 may also be provided to synchronise operation of the microprocessor, such as sampling of sensor data. A wireless transceiver 328 is provided to enable wireless communication of data from the microprocessor 316 to the remote station 104. Any suitable wireless communications protocol may be used. For example, the wireless transceiver 328 may be a LoRaWAN wireless transceiver module. The wireless transceiver 328 is coupled to the antenna 206 (via connector 338) to receive data from, and transmit data to, the remote station 104.

A further transceiver module 329 may be used to enable wired communications between the microprocessor 316 and an external device. The further transceiver module 329 may be an RS485 transceiver module. The external device may be any suitable mobile device, such as a laptop computer, smartphone and the like, that can be connected to the apparatus 200 via a wired connection via transceiver 329 (or wirelessly via transceiver 328) to download and/or upload data to/from the microprocessor 316.

The control circuit 201 further includes a battery management module 330. The battery management module 330 includes a battery monitoring unit 332 for monitoring the operation and performance of the battery cells 210; battery charging unit 334 for charging the battery cells 210 (for example via solar power 340 or another suitable external power source); and a power regulation unit 336 for regulating the power delivery from the battery cells 210 to the rest of the circuit module 300. A solar module 340 including one or more solar panels may be provided externally of the apparatus 200 for charging the battery cells 210 via solar power. Similarly to the sensor circuit 212, the power flow region 342 of the control circuit 201 is illustrated using dashed lines.

The configuration of an apparatus circuit module 350 according to another example embodiment is illustrated in FIG. 3B, where like features refer to those previously described with respect to FIG. 3A. The circuit module 350 functions similarly to the circuit module 300 previous described. The difference being that the circuit module 350 includes different sensor modules 352 to the sensor modules 516 of circuit module 300. In particular, the sensor modules 352 may include a broad-spectrum metal-oxide sensor 354. Sensor 354 may be configured to provide different resistance values indicative of concentrations of different gaseous species (e.g. any one or more of hydrogen, methane, helium, carbon monoxide, carbon dioxide and ammonia, or nitrogen dioxide in any combination). Moreover, sensor 354 may also provide additional resistance values indicative of environmental variables such as temperature, humidity and pressure. Sensor modules 352 further includes a chemiresistive (e.g. metal-oxide (MOX)) hydrogen sensor 356, a chemiresistive methane sensor 358, a chemresistive multi-gas sensor 360, and an electrolyte hydrogen sensor 362. In practice, any one or more of the sensors 354, 356, 358, 360, 362 may be combined in one or more integrated sensor units or provided as separate sensors. The specific combination of sensors is not limited to those described herein and any suitable number of sensors may be used in any suitable combination based on specific application requirements.

Sensor data from the sensor modules 352 are retrieved and processed in the same manner as that previously described with reference to the circuit module 300 of FIG. 3A.

A further schematic of a gas sensing apparatus 230 according to another example embodiment is illustrated in FIG. 3C. The gas sensing apparatus 230 functions in a similar manner to the gas sensing apparatus 200 described herein. Like features of the gas sensing apparatus 230 refer to those described herein with reference to the gas sensing apparatus 200 and circuit modules 300, 350. In particular, the gas sensing apparatus 230 may include all components and features of gas apparatus 200 and circuit modules 300 and 350.

In addition, the gas sensing apparatus 230 may further include a pump 232. The pump 232 may be internal or external to the housing 202. In the specific embodiment shown in FIG. 3C, the pump 232 is position within the housing 202 and configured to extract gases from the cavity 214. In some embodiments, the pump 232 may be configured to extract gases from internal spaces of the housing 202. Moreover, the pump 232 may flush the cavity 214 or internal spaces of the housing 202 with atmospheric gases (e.g. retrieved externally of the housing 202). In some embodiments, the apparatus 230 may include a compressed gas source 235 for flushing the cavity 214 or internal spaces of the housing 202. When using a compressed gas source 235, the pump 232 may not be required and activating the valve 240 may enable reference gas from the compressed gas source 235 to enter the cavity 214 during flushing operations.

In the embodiment shown, the apparatus 230 may include an outflow conduit 234 and an inflow conduit 236 for providing gas flow paths out of and into the cavity 214. A valve 238, 240 may be coupled to each of the two conduits 234, 236. The value 240 may be a three-way solenoid valve. Valve 238 may be a two-way solenoid valve.

The apparatus 230 may be configured to enable gas flux measurements as described in further detail below. Typically, prior to the determination of gas flux, gases in the cavity 214 may be flushed with a reference/atmospheric gas, so that a change of concentration of a particular gaseous species of interest over time may be determined more accurately.

During flushing operations, the pump 232 may be activated to pump gases from the atmospheric gases 242 into the cavity 214. The inlet 244 of inflow conduit 236 may be in fluid communication with the atmosphere such that atmospheric gases 242 may enter the cavity 214 via the inlet 244 during operation of the pump 232. Alternatively, or in combination, reference gas from the compressed gas source 235 may flow into the cavity 214 upon activation of valve 240. As gases flow into cavity 214, existing gases in the cavity 214 flow out of the cavity 214 and are therefore ‘flushed’ out via outlet flow path provided by outflow conduit 234.

The pump 232 may be powered by battery units 210 in the apparatus 230 and controlled via microprocessor 316 to operate at predetermined time intervals to enable flux calculations. This will be described in further detail below with reference to FIG. 3D. In one example, the microprocessor 316 may be operatively configured to operate the pump for 10 seconds so as to flush the cavity 214 for 10 seconds.

The implementation of the gas sending apparatus 230 including the pump assembly 232 enables determination of flux measurements using the apparatus 230.

Measurements for gas flux may be determined based on equation [1] below:

$$f ( gas ) = \frac{V}{A} \frac{Δ C}{Δ t} k \qquad [ 1 ]$$

wherein
f(gas) is gas flux having units in g^{m-2} t⁻¹,
V is the volume of the cavity in m³,
A is a total surface area of the barrier 208 in m²,
C is the concentration of a particular gaseous species of interest (e.g. H₂) measured (ppm, or g m⁻³) over time t in seconds s, and
k is a factor related to the gas permeability of the chosen membrane.

The determined values for gas flux f(gas) provides an indication of a rate at which a particular target gaseous species (e.g. H₂) is being emitted (e.g. from the soil). As mentioned, to achieve determination of gas flux f(gas), the apparatus 230 requires the ability to ‘purge’ or flush internal spaces of the housing 202 including the cavity 214 using a reference gas (e.g. from the compressed gas source 235) or atmospheric gas (e.g. via inflow conduit inlet 244), so as to enable determination of change in concentration measurements over time.

As barrier 208 reduces the efflux rate of gases (e.g. soil gases) into the cavity 214, a factor k is introduced to account for the effects of the barrier 208. Typically, a small cavity 214 size may minimise gas gradient effects and reduce volume of purge required for resetting flux measurement conditions. A lower limit of cavity size 214 may be determined by the maximum sampling rate to determine concentration of a particular gaseous species of interest (e.g. H₂) to calculate flux f(gas). In one embodiment, the sampling rate may be 1 to 2 samples per second.

A method of operating the pump 232 and valves 238, 240 as executed by the microprocessor 316 and calculation of flux according to one embodiment will now be described with reference to FIG. 3D. As mentioned, the microprocessor 316 controls operation of the pump 232 and valves 234, 240 to enable calculations of flux based on equation [1] above.

During normal operation of the apparatus 230, the microprocess 316 samples data from sensor modules 352 at a predetermined sampling frequency. For example, the microprocessor 316 may sample data from the sensor modules 352 at a sampling period of 1 sample every 10 seconds.

At step 412, the microprocessor 316 samples sensor data from the sensor modules 352 including data indicative of gaseous concentrations at the predetermine sampling frequency.

At query step 414, the microprocessor 316 determines whether a flux measurement is scheduled, for example based on a prior user configuration or a user request received via antenna 206 from an external user device and/or remote station 104. If so, the method 410 proceeds to query step 416. If not, the method 410 returns to step 412.

At query step 416, the microprocessor 316 may determine whether hydrogen concentration detected by sensor data from the sensor modules 352 is above a predetermined threshold (e.g. 100 ppm). In one embodiment, the microprocessor 316 may perform calibration of sensor data sampled from sensor modules 352 so as provide a prediction of actual hydrogen concentration present and compare the predicted valve for actual hydrogen concentration against a threshold hydrogen concentration. In another embodiment, sensor data sampled by the microprocessor 316 may be transmitted via the antenna 206 to a remote device or station 104 and calibration of the sampled raw sensor data provide a prediction of actual hydrogen concentration may be carried out remotely (e.g. not local) to the apparatus 230 by the remote device/station 104. In this scenario, the actual hydrogen concentration may be compared to a threshold value at the remote station 104, or transmitted back to the microprocessor 316 such the comparison between the actual hydrogen concentration may be compared to the threshold value by the microprocessor 316. If the predicted hydrogen concentration is greater than the threshold value, the method 410 proceeds to step 418. If not, the method 410 returns to step 412.

At step 418, the microprocessor 316 initiates operations to enable flux calculations. In particular, the microprocessor 316 activates the valves 240, 238 and/or the pump 232. The pump 232 pumps atmospheric gases into the cavity 214. In one embodiment, the valve 240 is a three-way valve and may permit gases from the compressed gas source 235 or inlet 244 to pass through the conduit 236 and into the cavity 214. In some applications, the apparatus 230 may not have access the atmospheric gases, for example if it is necessary to deploy the apparatus 230 underground. In these applications, three-way valve permits gas flow from the compressed gas source 235 into the cavity 214. As gases flow into the cavity 214 from inflow conduit 236, existing gases are displaced or flushed out of the cavity 214 via outflow conduit 234. As gas flux is a measurement of a change in the concentration of a particular gaseous species of interest over time, it is useful to flush the cavity 214 with reference or atmospheric gases prior to the calculation of flux to improve the accuracy of flux measurements.

At step 420, the microprocessor 316 may determine whether the current predicted actual concentration of hydrogen is below a lower threshold (e.g. 100 ppm). Similarly to step 416, the calibration of sensor data to predict hydrogen concentration and/or the comparison against the lower threshold may be performed on board the microprocessor or remotely (e.g. via a remote device/station 104 wirelessly connected to the microprocessor 316). If flushing operations have been successful, the actual hydrogen concentrations would typically drop below the lower threshold. If not, this may indicate that the flushing operation is incomplete or there may be a blockage in the gas flow path between and/or within the cavity 214 and the outflow conduit 234. If the predicted actual concentration of hydrogen is below the lower threshold or a predetermined time period expires, the method 410 proceeds to step 422. If not, the method 410 returns to step 418 and flushing operations continue.

At step 422, the microprocessor 316 determines that the flushing operations is either complete or cannot be completed due to an error (e.g. blockage). As such, the microprocessor 316 disables the valves 240, 238 and/or pump 232. The pump 232 is no longer moving gases into the cavity 214 and the valves 240, 238 are closed so as to prevent accumulated gases in the cavity 214 from escaping via the inflow and outflow conduits 236, 234.

At step 424, the microprocessor 316 samples the sensor data from the sensor modules 352 at a higher sampling frequency than the predetermined sampling frequency in step 412. For example, the elevated sampling frequency may be 2 samples per second.

At query step 426, the microprocessor 316 may determine whether the concentration of hydrogen in the cavity has stabilised based on the sampled sensor data from the sensor modules 352 in step 424. Similar to previous steps, this may be carried out locally on the microprocessor 316 or remotely. If it is determined that the concentration of hydrogen in the cavity has stabilised, the method 410 proceeds to step 428. If not, the method 410 returns to step 424.

At step 428, equation [1] can be used to calculate gas flux based on the change in hydrogen concentration within the cavity over time, the relevant time period being from the start of step 424 until a determination that the concentration of hydrogen has stabilised in step 426.

In some embodiments, the apparatus 200 and/or 230 may form part of a system 400 for autonomous remote monitoring of gas seeps, such as hydrogen seeps in soil. The system may include a plurality of apparatus 200, or apparatus 230, or a combination of apparatuses 200, 230.

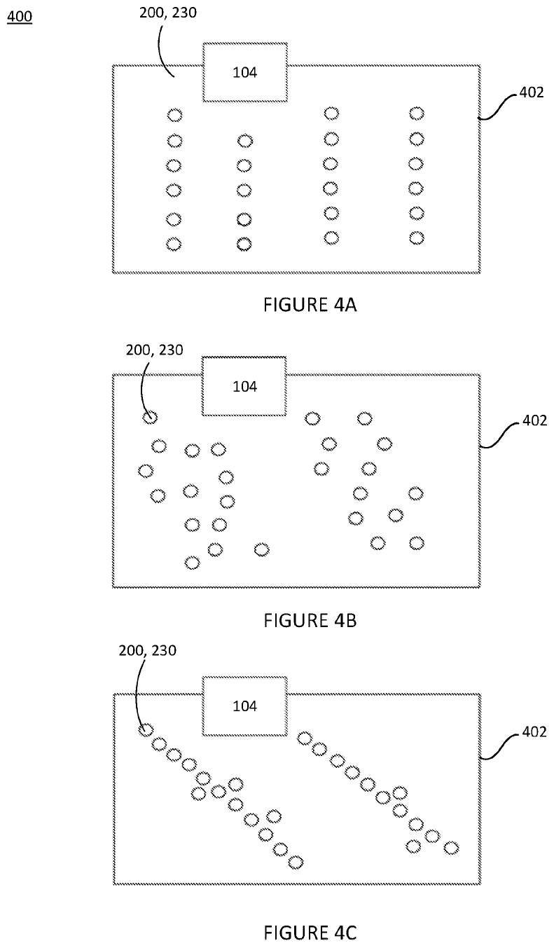

The system 400 may include a network of apparatuses 200 and/or 230 as illustrated in FIG. 4A to FIG. 4C. In practice, a plurality of apparatuses 200 and/or 230 may be evenly distributed over any area of interest for any suitable period of time. For example, a plurality of apparatuses 200 and/or 230 are distributed in a regular array pattern over an area of interest 402 as shown in FIG. 4A. Each apparatus 200 and/or 230 is inserted into the soil as shown in FIG. 1, and enabled for wireless communication with the remote station 104.

Advantageously, the network apparatuses 200, 230 can simultaneously and autonomously detect hydrogen seeps across the entire area 402 so that hydrogen concentrations can be more effectively and conveniently determined and monitored.

In the field of environmental monitoring, hydrogen seeps can occur naturally in areas such as geothermal fields and hydrothermal vents, or as a result of human activity such as oil and gas exploration. The release of hydrogen gas into the environment can have ecological impacts, such as changes to microbial communities and the potential for explosion hazards. Enabling remote, autonomous and continuous monitoring of hydrogen concentrations over a larger area 402 can help to more accurately and effectively identify the extent and impact of hydrogen seeps on the environment.

In the field of resource exploration, hydrogen seeps can also be an indicator of underlying resources, such as oil and gas deposits or geothermal reservoirs. Enabling remote, autonomous and continuous monitoring of hydrogen concentrations over a larger area 402 can also help to more accurately and effectively identify the location and extent of these resources, potentially leading to new exploration and development opportunities.

As shown in FIG. 4B, the positioning of each apparatus 200, 230 within the network can also be altered based on detected concentrations of hydrogen after an initial deployment phase (e.g. as shown in FIG. 4A). In FIG. 4B, the specific locations of each apparatus 200, 230 is adjusted to areas of higher detected concentrations of hydrogen. After a period of deployment in the positions shown in FIG. 4B, the locations of each apparatus 200, 230 may be further adjusted as shown in FIG. 4C so that each apparatus 200, 230 is moved even closer to locations where the highest concentrations of hydrogen are detected. The positioning of each apparatus 200, 230 may be adjusted any suitable number of times over any suitable number of iterations until one or more concentrated areas within the larger area of interest 402 where the highest concentration of hydrogen seeps can be identified, so as to more accurately determine the specific locations of one or more potential sources of the hydrogen seeps.

Once collected, sensor data from the sensors 216, 352 is calibrated to determine a concentration of each of the detected gaseous species, for example including any one or more of hydrogen (H₂), methane (CH₄), helium (He), carbon monoxide (CO), carbon dioxide (CO₂), ammonia (NH₃), and nitrogen dioxide (NO₂), and any other suitable gaseous species of interest.

In one embodiment, the calibration of sensor data to determine respective concentrations of each detected gaseous species can be performed on board each apparatus 200, 230 by the respective microprocessor 316, and the calibrated gas concentration data can be transmitted to the remote station 104.

In one embodiment, the calibration of sensor data to determine respective concentrations of each detected gaseous species for each apparatus 200, 230 in a network of apparatuses 200, 230 can be performed at the remote station 104. In these embodiments, raw sensor data may be transmitted from each apparatus 200, 230 to the remote station 104.

In some embodiments, the remote station 104 and the microprocessor 316 of each apparatus 200, 230 may both be configured to perform the calibration of sensor data. In some embodiments, more than one remote station may be provided.

The calibration of raw sensor data to determine concentrations of respective gaseous species can be carried out in a number of different ways. For example, a prediction model may be derived to determine gas concentrations. In some embodiments, a single prediction model may be derived to determine concentrations of all gaseous species detected. In some embodiments, one or more prediction models may be derived, each prediction model for determining the concentrations of any one or more of the detected gaseous species. Whilst a number of the example embodiments herein describe the detection of hydrogen (H₂) and the calibration to determine the concentration of hydrogen (H₂) as a target gaseous species of interest, a skilled addressee would understand that the inventive concept may be similarly applied to detect and determine the concentration of any target gaseous species of interest, for example any one or more of hydrogen (H₂), methane (CH₄), helium (He), carbon monoxide (CO), carbon dioxide (CO₂), ammonia (NH₃), and nitrogen dioxide (NO₂), and any other suitable gaseous species of interest based on the application.

The prediction model may be derived using statistical models such a linear regression model to determine the actual gas concentrations based on raw sensor data. To derive the prediction model based on linear regression, experiments are set up to determine the sensor resistance response (e.g. in Ohms or Volts) for each one of the four sensors 216 when the sensors 216 are exposed to known concentrations of each gaseous species hydrogen (H₂), methane (CH₄), helium (He), carbon monoxide (CO), carbon dioxide (CO₂), ammonia (NH₃), and nitrogen dioxide (NO₂). Experimental data is then used to derive the linear regression prediction model.

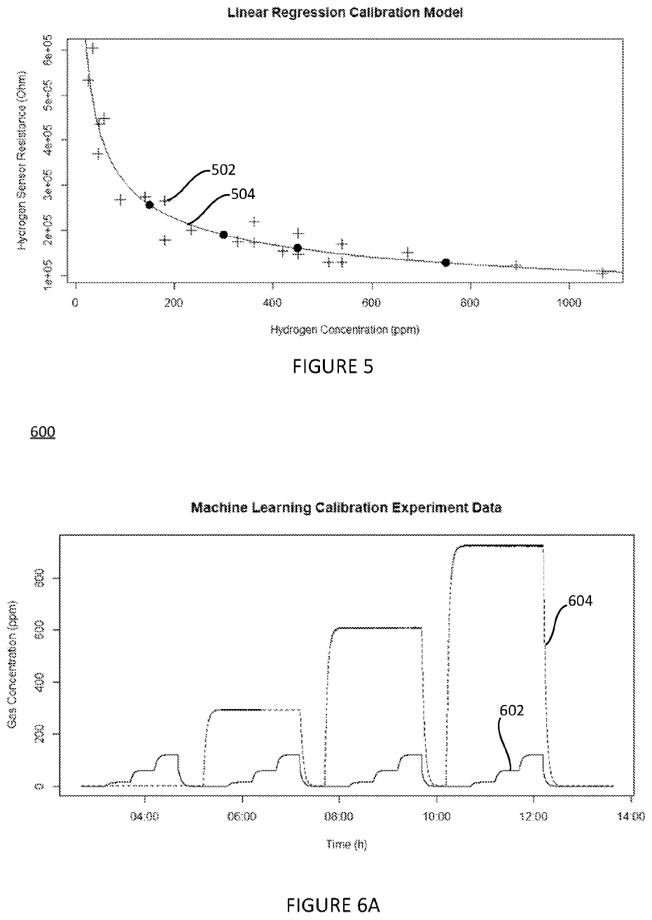

An example linear regression prediction model 500 for hydrogen is shown in FIG. 5. Log values of output sensor resistance for the hydrogen gas sensor 302 and log values of corresponding actual hydrogen gas concentrations are used to fit the linear regression model. In FIG. 5, data points 502 represent measured sensor resistance values (ohm) from the hydrogen gas sensor 302 (y-axis) against known hydrogen concentrations (ppm) (x-axis) in a controlled environment during performance testing. The fitted curve 504 is the derived linear regression prediction model to determine a concentration of hydrogen (ppm) based on a measured sensor resistance value (ohm) using the hydrogen gas sensor 302.

In some embodiments, the prediction model may be derived using machine learning, for example by using supervised machine learning models including gradient boosting machine learning, or random forest algorithms.

The supervised machine learning model is pre-trained based on sensor data indicative of concentrations of hydrogen (H₂) and one or more gaseous species other than hydrogen (H₂), and actual gas concentrations of hydrogen (H₂) and one or more gaseous species other than hydrogen (H₂) corresponding to the sensor data.

To obtain the training data required to train the machine learning model, experiments are set up in a controlled environment to measure sensor response when exposed to varying known concentrations of different gases.

Example training and experimental data obtained from one such experiment is shown in FIGS. 6A and 6B. During the example experiment, apparatus 200 is placed in an environmental chamber in which 1 L/min of constant air flow passes through the chamber. During the experiment, the apparatus 200 is exposed to different concentrations of hydrogen and methane in a staged manner.

During a first stage, the apparatus 200 is exposed to 15, 60, and 120 ppm of hydrogen for 600 s at each of these three known hydrogen concentrations. After a 600 s break, the same hydrogen concentrations (15, 60, and 120 ppm) are provided again for 600 s at each concentration in the presence of 300 ppm of methane during a second stage. After another 600 s break, the same hydrogen concentrations are provided again for 600 s at each concentration in the presence of 600 ppm of methane during a third stage. After another 600 s break, the same hydrogen concentrations are provided again for 600 s at each concentration in the presence of 900 ppm of methane during a fourth stage. The experiment may be repeated, and with different concentrations of hydrogen and methane at consecutive stages.

In FIG. 6A, line 602 represents actual concentrations of hydrogen within the environmental chamber over time during the experiment, and line 604 represents the actual concentrations of methane within the environmental chamber over time during the experiment. It has been observed that the actual gas concentration values in the environmental chamber lags the known delivered gas concentration values. This is because time is required for the gases to enter the chamber. The actual gas concentration value curves 602, 604 shown in FIG. 6A may be obtained by measuring the gas concentration of at least one of methane or hydrogen in the environmental chamber using an independent gas sensor.

In one example, the actual gas concentration of methane 604 in the environmental chamber is measured by an independent sensor. The measured actual gas concentration of methane 604 can be used to determine a change of actual methane concentration in the environmental chamber when a known concentration of methane is initially delivered to the environmental chamber. The determined change characteristic of actual methane concentrations can be used to estimate a change of actual hydrogen concentrations in the environmental chamber when a known concentration of hydrogen is initially delivered to the environmental chamber, without the need to take measurements using an independent hydrogen sensor. In another example, one or more independent sensors may be used to determine the actual gas concentrations for hydrogen and methane in the environmental chamber. Typically, such independent sensors may be high precision instrumentation which are expensive and relatively large in size, and therefore not suitable for mass deployment in the field.

FIG. 6B illustrates raw sensor data indicative of hydrogen (H₂) 608, nitrogen dioxide (NO₂) 610, carbon monoxide (CO) 612, and ammonia (NH₃) 614 concentrations taken from the hydrogen gas sensor 302 and thermoconductive gas sensor 306 during the same experiment.

In one example, a training dataset can then be compiled using the actual concentrations of hydrogen 602 as shown in FIG. 6A and the corresponding sensor response 608 from the hydrogen gas sensor 302. The training dataset can then be used to train the machine learning model to determine a concentration of hydrogen (ppm) for a given sensor output from a corresponding hydrogen gas sensor 302.

In other examples, training datasets can be compiled using the actual concentrations of hydrogen 602 and methane 604 as shown in FIG. 6A and the corresponding sensor response 608 from the hydrogen gas sensor 302 and methane gas sensor (not shown). In yet further examples, training datasets can be compiled using actual concentrations of any one or more gaseous species of interest, and the corresponding sensor responses from the respective gas sensors in a similar manner. The training dataset can then be used to train the machine learning model to determine a concentration of hydrogen (ppm) for a given sensor output from a corresponding hydrogen gas sensor 302, and the concentration of any one or more other gaseous species based on raw gas sensor data.

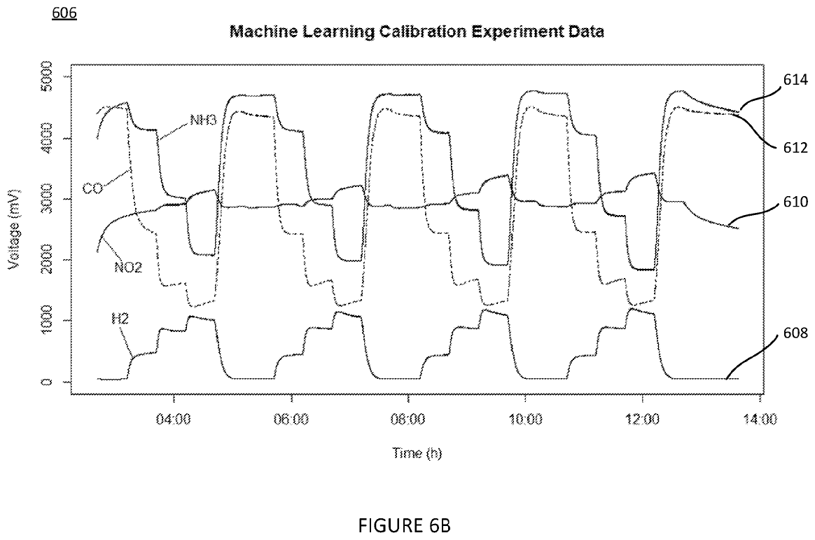

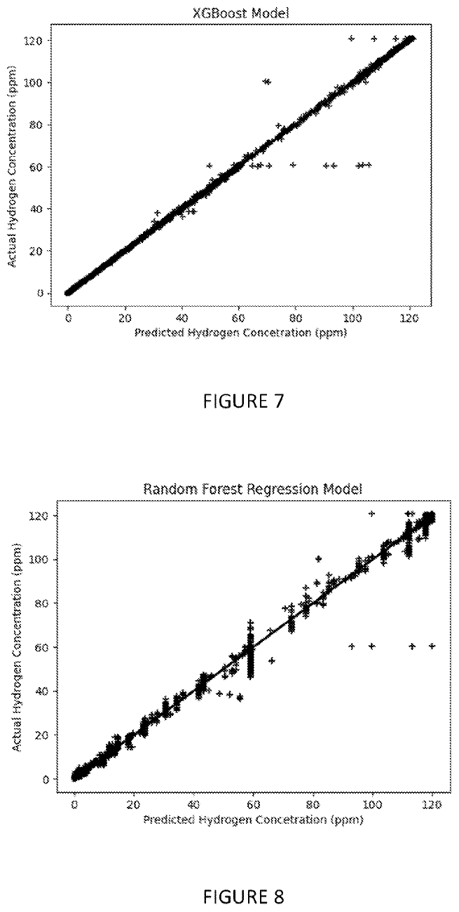

FIG. 7 is a scatter plot of actual (known) hydrogen concentrations (ppm) against predicted hydrogen concentrations (ppm) using a trained gradient boosting machine learning model, illustrating the accuracy of the trained gradient boosting machine learning model for calibrating sensor data to determine hydrogen concentrations.

FIG. 8 is a scatter plot of actual (known) hydrogen concentrations (ppm) against predicted hydrogen concentrations (ppm) using a trained random forest regression model, illustrating the accuracy of the trained random forest regression model for calibrating sensor data to determine hydrogen concentrations.

Similar experiments can be set up in a controlled environment in which known concentrations of hydrogen (H₂), methane (CH₄), helium (He), carbon monoxide (CO), carbon dioxide (CO₂), ammonia (NH₃), and nitrogen dioxide (NO₂), and environmental conditions including temperature, humidity and pressure are varied in a staged manner to generate an appropriate training data set to train a supervised machine learning model to determine the concentration of hydrogen, methane (CH₄), helium (He), carbon monoxide (CO), carbon dioxide (CO₂), ammonia (NH₃), and nitrogen dioxide (NO₂) in a gas mixture under specific or varying atmospheric or environmental conditions.

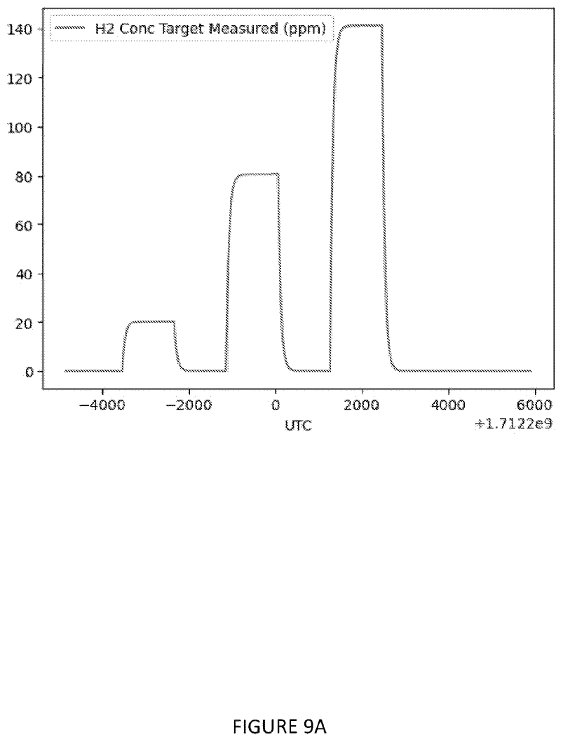

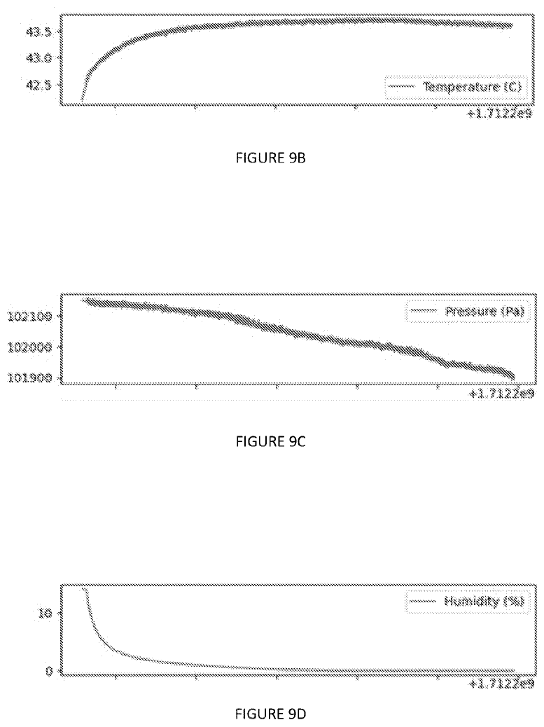

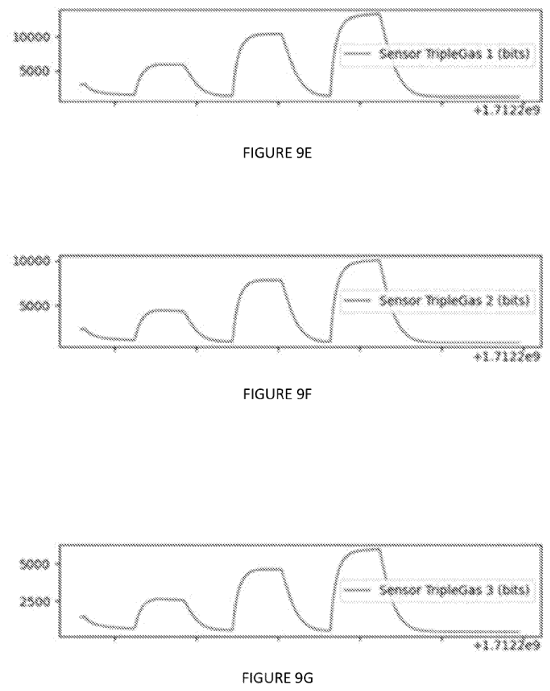

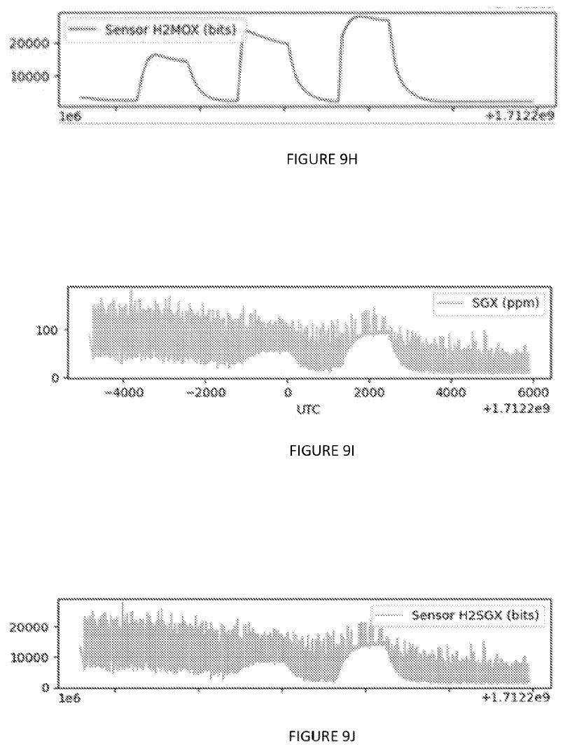

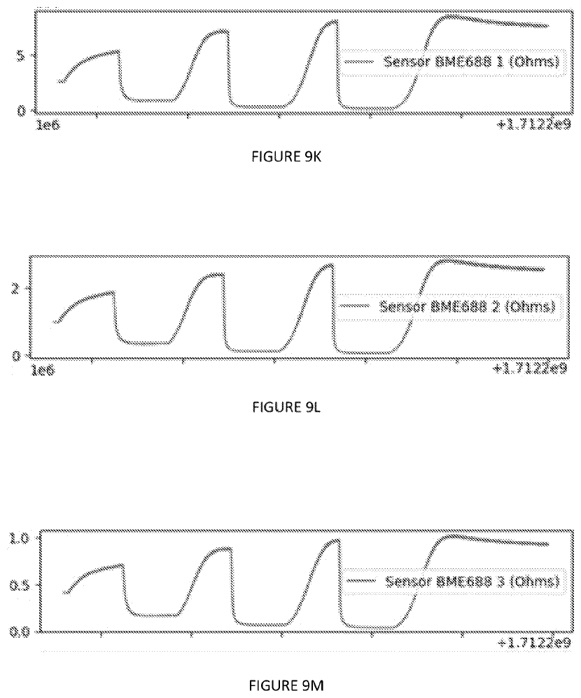

Example training and experimental data obtained from another experiment is shown in FIGS. 9A to 9M. During the example experiment, apparatus 200, 230 may be placed in an environmental chamber similar to that previously described. During the experiment corresponding to FIGS. 9A to 9M, the apparatus 200,230 is exposed to different concentrations of hydrogen in a staged manner in the environmental chamber.

As illustrated in FIG. 9A, the apparatus 200, 230 is exposed to a varied concentration of hydrogen at predetermined time intervals. The x-axis of FIG. 9A represents time in UTC measured in seconds, and the y-axis represents gas concentration in ppm. In particular, FIG. 9A illustrates that during the experiment, the apparatus 200, 230 was exposed to 0 ppm, 20 ppm, 0 ppm, 80 ppm, 0 ppm and 140 ppm of hydrogen at 20-minute intervals.

The apparatus 200, 230 may include the following sensor modules 352 as illustrated in FIG. 3B:

Sensor unit 1 being a metal-oxide (MOX) hydrogen sensor (illustrated as sensor 356 in circuit module 350 of FIG. 3B)

Sensor unit 2 being an electrolyte hydrogen sensor (illustrated as sensor 362 in circuit module 350)

Sensor unit 3 being a triple sensor sensitive to hydrogen, methane, and carbon-monoxide (illustrated as sensors 358 and 360 in circuit module 350). The output of sensor unit 3 provides three measurements of resistance, each measurement of resistance being sensitive to the presence of hydrogen, methane, and/or carbon-monoxide.

Sensor unit 4 being a broad-spectrum metal-oxide sensor (illustrated as sensor 354 in circuit module 350). Sensor unit 4 provides three different resistance values each measurement of resistance being sensitive to the presence of different gaseous species such as hydrogen and volatile organic compounds (VOC). Sensor unit 4 also provides resistance values sensitive to changes in environmental conditions including temperature, humidity and pressure in the cavity 214.

The output of the experimental sensor data from each of the four sensor units above is illustrated in FIGS. 9B to 9M. In particular,

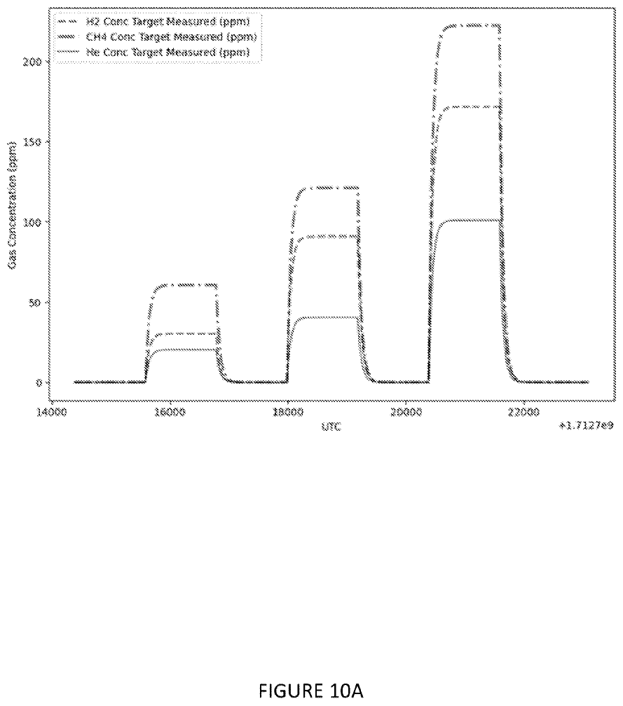

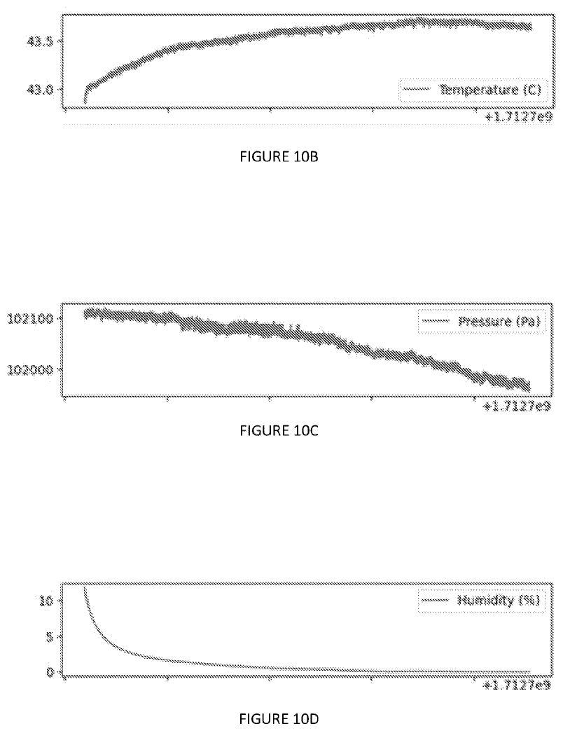

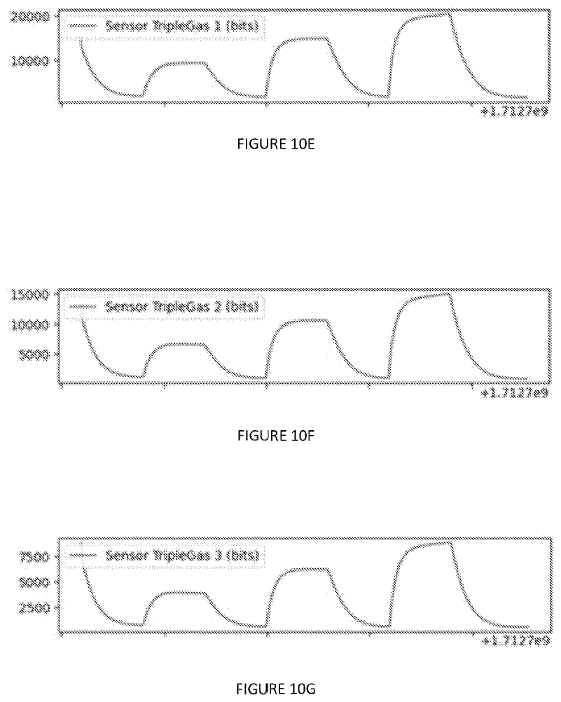

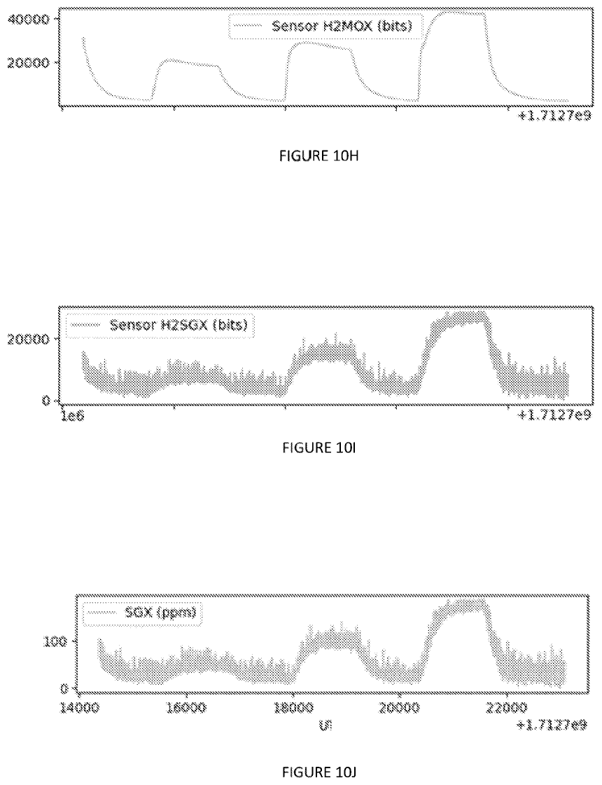

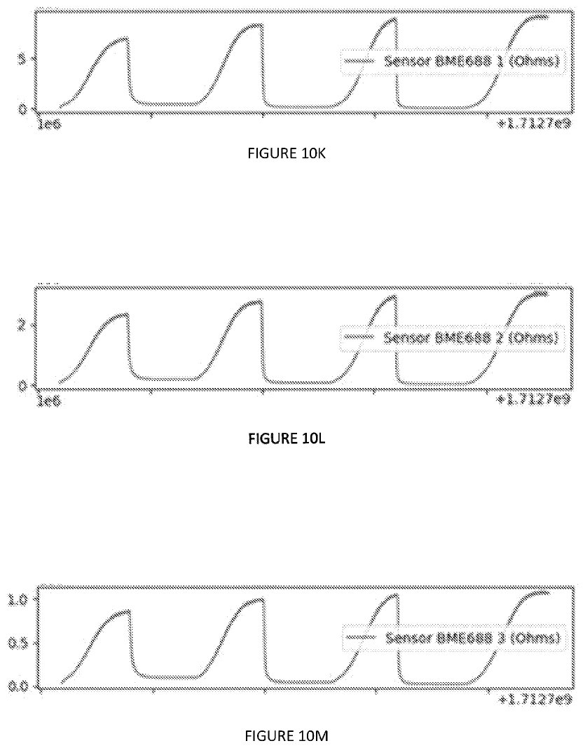

FIG. 9B illustrates sensor output from sensor unit 4 indicative of temperature variation in the cavity 214 of the apparatus 200, 230 (corresponding sensor output for further experiments as described below are also illustrated in FIGS. 10B, 11C, 12A).

FIG. 9C illustrates sensor output from sensor unit 4 indicative of pressure variation in the cavity 214 of the apparatus 200, 230 (corresponding sensor output for further experiments as described below are also illustrated in FIGS. 10C, 11D, 12B).

FIG. 9D illustrates sensor output from sensor unit 4 indicative of humidity variation in the cavity 214 of the apparatus 200, 230 (corresponding sensor output for further experiments as described below are also illustrated in FIGS. 10D, 11E, 12C).

FIG. 9E illustrates sensor output in bits from a first gaseous sensor of sensor unit 3 (corresponding sensor output for further experiments as described below are also illustrated in FIGS. 10E, 11F, 12D).

FIG. 9F illustrates sensor output in bits from a second gaseous sensor of sensor unit 3 (corresponding sensor output for further experiments as described below are also illustrated in FIGS. 10F, 11G, 12E).

FIG. 9G illustrates sensor output in bits from a third gaseous sensor of sensor unit 3 (corresponding sensor output for further experiments as described below are also illustrated in FIGS. 10G, 11H, 12F).

FIG. 9H illustrates sensor output in bits from the hydrogen sensor of sensor unit 1 (corresponding sensor output for further experiments as described below are also illustrated in FIGS. 10H, 11I, 12G).

FIG. 9I illustrates sensor output in ppm from the hydrogen sensor of sensor unit 1 (corresponding sensor output for further experiments as described below are also illustrated in FIGS. 10I, 11J, 12H).

FIG. 9J illustrates sensor output in ppm from the hydrogen sensor of sensor unit 2 (corresponding sensor output for further experiments as described below are also illustrated in FIGS. 10J, 11K, 12I).

FIG. 9K illustrates sensor output in ohms from a first gaseous sensor of sensor unit 4 (corresponding sensor output for further experiments as described below are also illustrated in FIGS. 10K, 11L, 12J).

FIG. 9L illustrates sensor output in ohms from a second gaseous sensor of sensor unit 4 (corresponding sensor output for further experiments as described below are also illustrated in FIGS. 10L, 11M, 12K).

FIG. 9M illustrates sensor output in ohms from a third gaseous sensor of sensor unit 4 (corresponding sensor output for further experiments as described below are also illustrated in FIGS. 10M, 11N, 12L).

As illustrated in FIGS. 9E to 9M, all sensing elements in the gaseous sensors exhibit sensitivity to changes in the concentration of hydrogen introduced into the environmental chamber, regardless of the manufacturer's indication of the specific gaseous species a particular sensing element is designed to detect.

As illustrated in FIG. 10A, the apparatus 200, 230 is exposed to varied concentrations of hydrogen, methane and helium at predetermined time intervals. Again, the x-axis of FIG. 10A represents time in UTC measured in seconds, and the y-axis represents gas concentration in ppm. In particular, FIG. 10A illustrates that during the experiment, the apparatus 200, 230 was exposed to the following gas concentration variations concurrently at 20-minute intervals:
0 ppm, 30 ppm, 0 ppm, 90 ppm, 0 ppm and 170 ppm of hydrogen (H₂)
0 ppm, 60 ppm, 0 ppm, 120 ppm, 0 ppm and 220 ppm of methane (CH₄)
0 ppm, 20 ppm, 0 ppm, 40 ppm, 0 ppm and 100 ppm of helium (He)

The output of the experimental sensor data from each of the four sensor units is illustrated in FIGS. 10B to 10M, wherein each of the FIGS. 10B to 10M correspond to a respective one of the sensor output description previously set out above in relation to FIGS. 9B to 9M.

As illustrated in FIGS. 10E to 10M, all sensing elements in the gaseous sensors exhibit sensitivity to changes in the concentration of gases introduced into the environmental chamber as illustrated in FIG. 10A. However, each of the sensor units 1 to 4 exhibit different responses (in output amplitude and shift) to the change in concentration of the mixture of cross-contaminating gases. It has also been noted that the output of sensor unit 1 (electrolyte hydrogen sensor) is noisy when exposed to low hydrogen concentrations (e.g. below 200 ppm). As such, a filter may be used to filter out the noisy sensor signal below a threshold gas concentration (e.g. <200 ppm). Sensor data as illustrated in FIGS. 10B to 10M provide an indication of sensor output behaviour when the sensor units 1 to 4 are exposed to a mixture of cross-contaminated gases in varying concentrations.

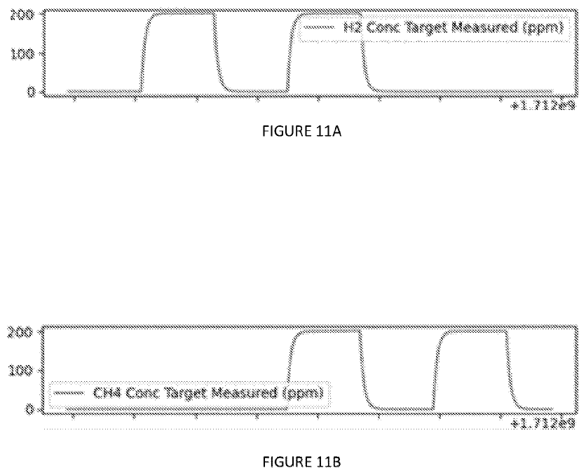

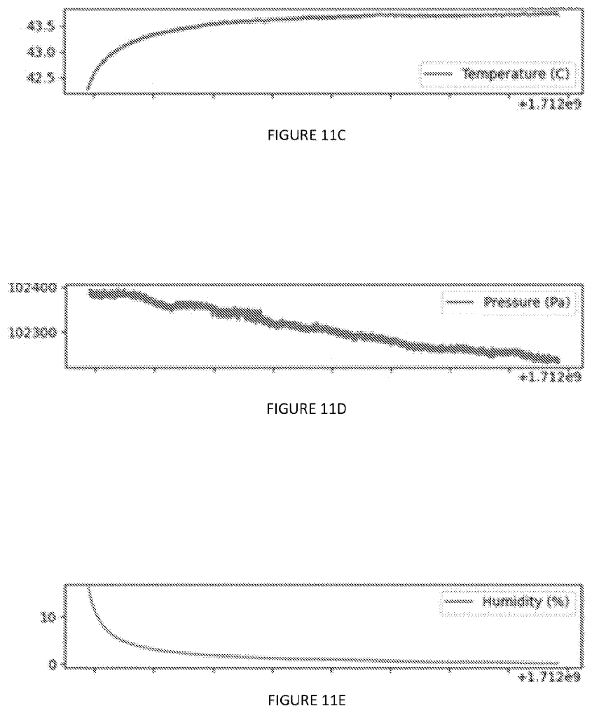

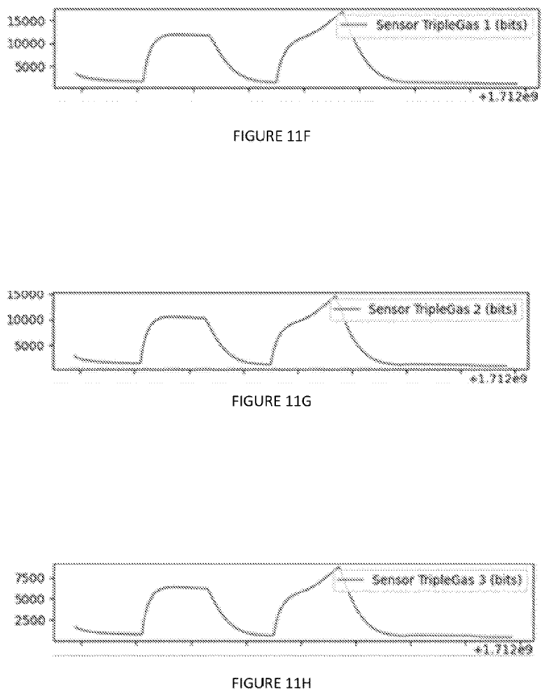

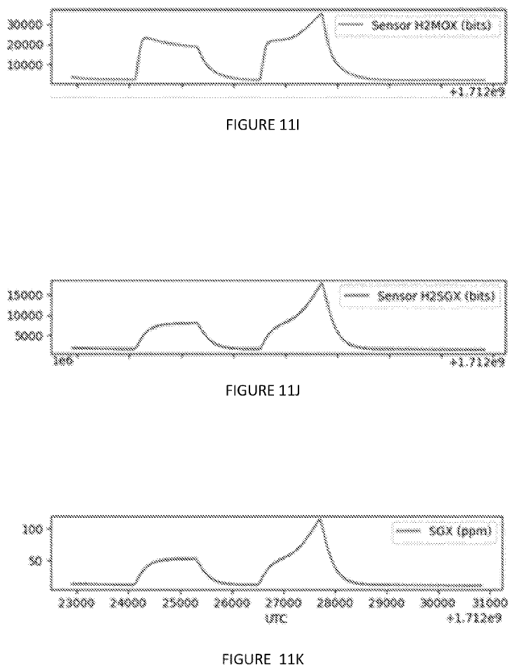

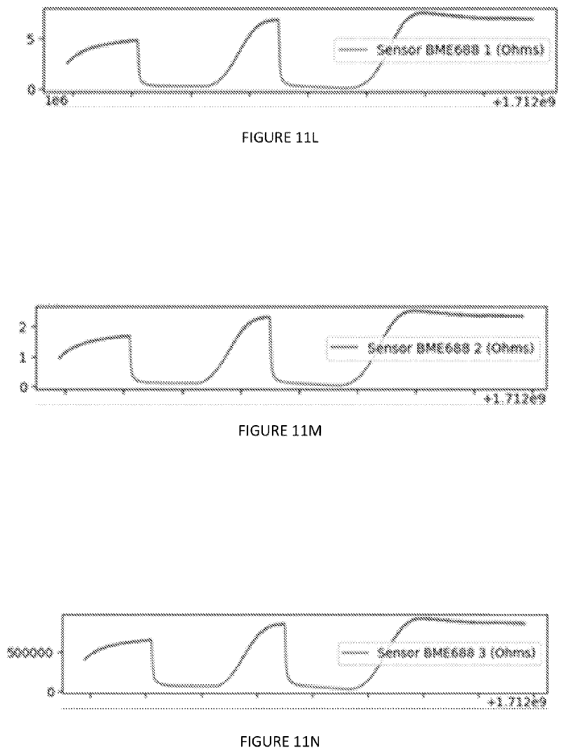

FIGS. 11A and 11B illustrate a further experiment whereby the apparatus 200, 230 is exposed to varied concentrations of hydrogen and methane individually and at the same time to determine the effect on sensor responses. The x-axis of each of FIGS. 11A and 11B represents time, and the y-axis represents gas concentration in ppm. In particular, FIGS. 11A and 11B illustrates that during the experiment, the apparatus 200, 230 was exposed the following gas concentration variations concurrently at predetermined time intervals:
0 ppm, 200 ppm, 0 ppm, 200 ppm, 0 ppm, 0 ppm of hydrogen (H₂)
0 ppm, 0 ppm, 0 ppm, 200 ppm, 0 ppm and 200 ppm of methane (CH₄)

The output of the experimental sensor data from each of the four sensor units is illustrated in FIGS. 11C to 11M, wherein each of the FIGS. 11C to 11M correspond to a respective one of the sensor output description previously set out above in relation to FIGS. 9B to 9M. It can be seen from the sensor output response that all gaseous sensing elements of the sensor units are affected by the cross contamination of methane with hydrogen. Based on the sensor output shown in FIG. 11I, sensor unit 1 is less sensitive to methane when compared with the other sensors.

Similar experiments to those described herein (e.g. with respect to 10A, 11A, 11B) may be carried out using any suitable combination of different gaseous species in any suitable concentrations to observe the behaviour of the sensor responses when exposed to different gaseous species at different combinations.

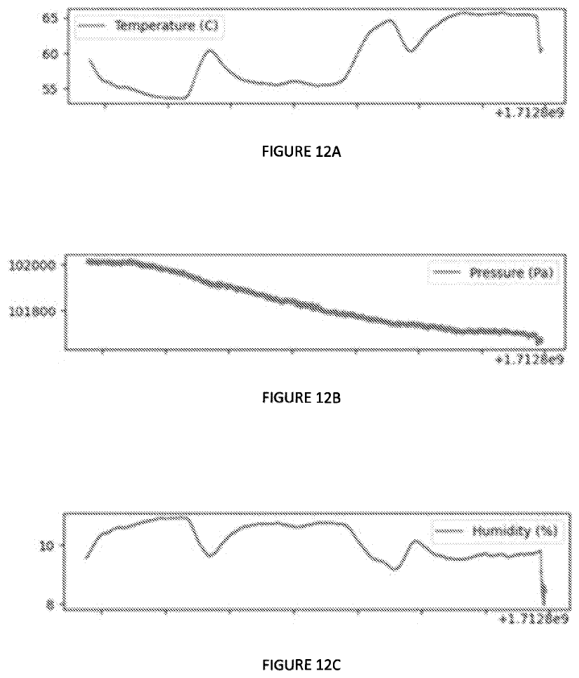

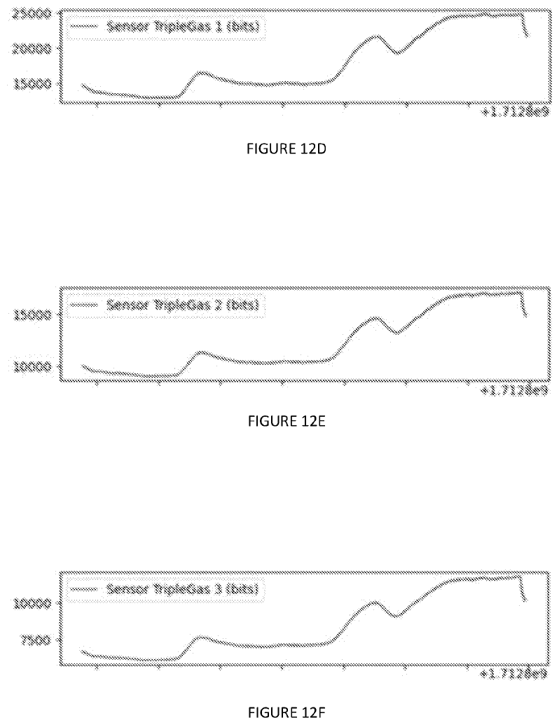

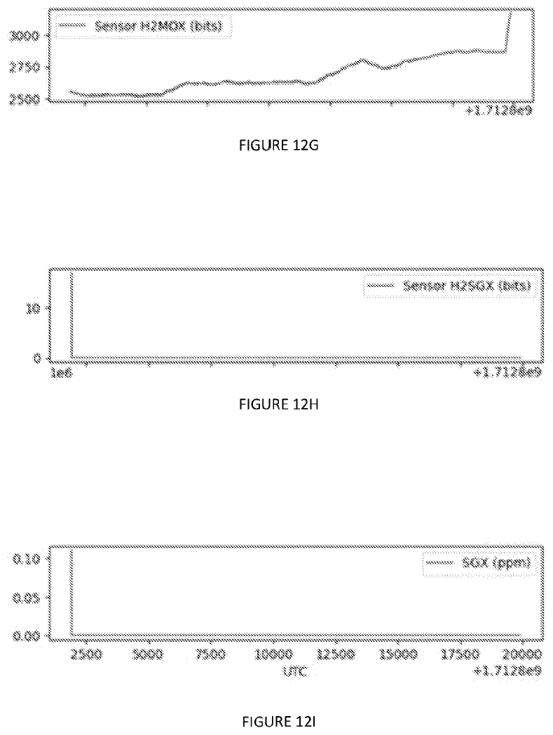

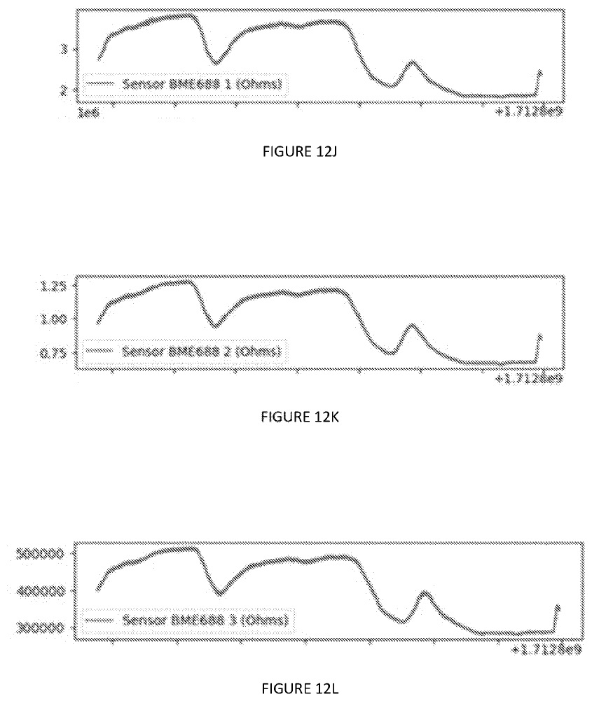

A further experiment as illustrated in FIGS. 12A to 12L is carried out to observe the impact on sensor output in varying environmental conditions, such as varying temperature, pressure and humidity conditions. FIGS. 12A to 12L illustrate sensor data collected using the apparatus 200, 230 when the apparatus 200, 230 is deployed outdoors at a testing site (e.g. the apparatus may be inserted into soil as illustrated in FIG. 1A). In this experiment, it may be verified using alternative instrumentation that no other gaseous species of interest are present at the testing site and that all of sensor responses observed are purely due to changes in ambient temperature (FIG. 12A), ambient pressure (FIG. 12B) and ambient humidity (FIG. 12C). In this experiment, the sensor data collected facilitates the machine learning and/or statistical models to learn correlation between variations in environmental conditions and sensor output response.

As described in further detail below, the sensor response data collected in the different experiments (e.g. as illustrated above in FIGS. 9A to 9I, 10A to 10M, 11A to 11N, 12A to 12L) may be used as training and validation datasets for machine learning and/or statistical models for calibration and estimation of a concentration of one or more particular gaseous species of interest, such as hydrogen, methane, helium and carbon dioxide, in the similar manner as previously described.

The training data set compiled using the experiments described herein may enable effective calibration and estimation of a concentration of one or more gaseous species of interest and correcting for the effects of cross contamination of different gaseous species, and environmental bias (e.g. due to varying environmental conditions). As mentioned, any suitable machine leaning and/or statistical models may be used to predict the concentration of one or more particular gaseous species of interest. In one embodiments, a gradient boosting algorithm (e.g. XGBoost) may be used.

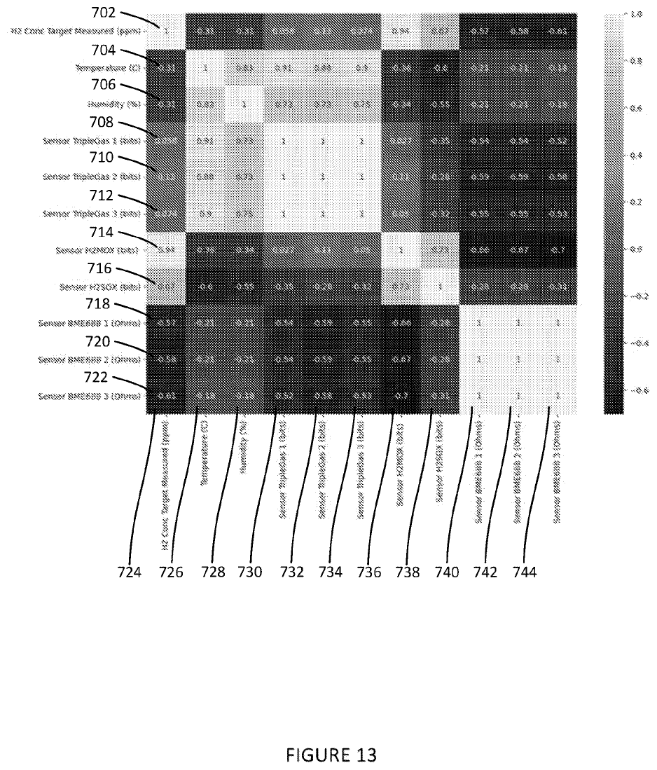

FIG. 13 is a correlation matrix of the variables illustrated in FIGS. 9A to 12L used in training a machine learning model. Each of the values in a cell of the matrix provides an indication of the correlation between the variables listed in a corresponding row and column of that cell. Typically, a value between about 0.75 and 1 indicates strong correlation, a value between about 0.6 to 0.75 indicates good correlation, and a value between 0.5 to 0.6 indicates moderate correlation, and a value below 0.5 indicates no correlation.

In particular, details of the variables represented by each row and column are listed below:
Row 702 and column 724 represent the concentration of target measured concentration of hydrogen (e.g. in ppm)
Row 704 and column 726 represent temperature
Row 706 and column 728 represent humidity
Row 708 and column 730 represent sensor output from a first gaseous sensor of sensor unit 3
Row 710 and column 732 represent sensor output from a second gaseous sensor of sensor unit 3
Row 712 and column 734 represent sensor output from a third gaseous sensor of sensor unit 3
Row 714 and column 736 represent hydrogen sensor of sensor unit 1
Row 716 and column 738 represent hydrogen sensor of sensor unit 2
Row 718 and column 740 represent sensor output from a first gaseous sensor of sensor unit 4
Row 720 and column 740 represent sensor output from a second gaseous sensor of sensor unit 4
Row 722 and column 744 represent sensor output from a third gaseous sensor of sensor unit 4

The matrix of FIG. 13 illustrates that the sensors that are most selective and responsive to concentrations of hydrogen based on the experimental data. In particular, based on in the experiments described herein, it may be determined that the hydrogen sensor of sensor unit 1 is most selective to hydrogen (see cell (702, 736)), following by the hydrogen sensor of sensor unit 2 (see cell (702, 738)).

In some embodiments, the apparatus 200, 230 may include two or more sensors specifically adapted to detect a particular gaseous species of interest such as hydrogen. In the example embodiments described above, two different hydrogen sensors may be provided in the apparatus 200, 230. These different sensor types may exhibit distinct responses to factors like temperature, humidity, and the presence of other gases. Moreover, each sensor may be better suited to detect hydrogen at different concentrations. By using sensor data from both of the two different hydrogen sensors, their complementary properties may be leveraged to improve accuracy and reliability of the trained machine learning model to detect hydrogen concentrations.

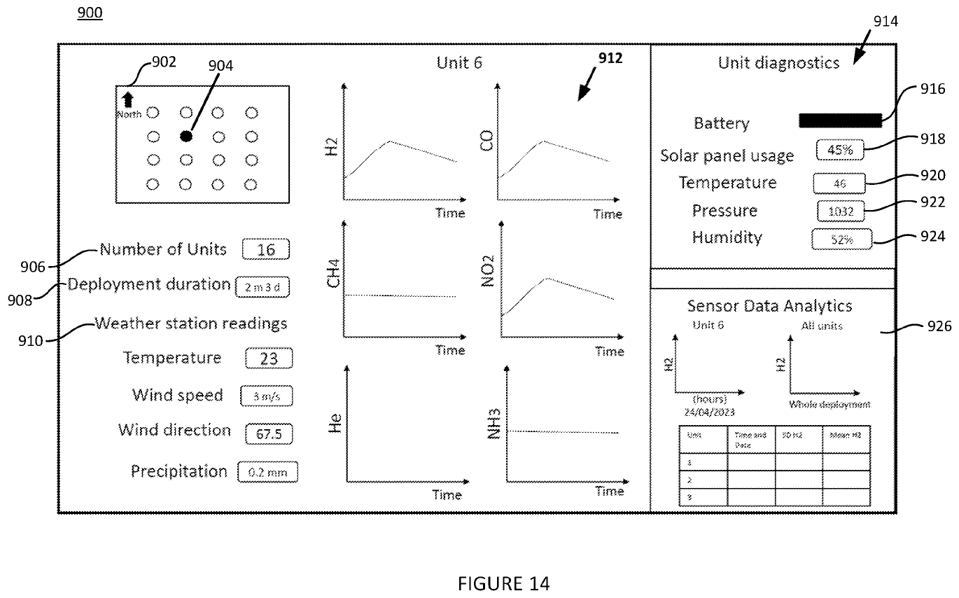

The remote station 104 may include a graphical user interface for displaying the calibrated gas concentration data, numerically and/or graphically in real time or near real time. An example graphical user interface 900 is illustrated in FIG. 14. In this embodiment, the interface 900 illustrates the deployment of a plurality of apparatus 200, 230 in the field 902 and allows selection of any one of the apparatuses 200, 230 for detailed analysis. In the example shown, the selected apparatus 904 is highlighted. The interface 200, 230 also displays general information regarding the deployed system 400, including the total number of apparatuses 200, 230 deployed 906, and the deployment duration 908. General weather information of the deployment site may also be obtained by the remote station from a weather station (not shown) for display via the graphical user interface 900. For example, weather information 910 pertaining to temperature, wind speed, wind direction, and precipitation may be displayed. Calibrated gas concentration data for each selected apparatus 904 may also be displayed graphically 912 on the interface 900. Moreover, diagnostic information 914 of the selected apparatus 904 may also be displayed. The diagnostic information 914 may include battery usage 916, solar power usage 908, and atmospheric sensor data including temperature 920, pressure 922 and humidity 924 from the respective atmospheric sensor 308. Specific gas sensor data analytics 926 for each detect gaseous species (e.g., hydrogen H₂) may be displayed to provide a more detailed analysis of the measured gas concentration. In the example shown in FIG. 14, the gas sensor analytics 926 is currently illustrating information relating to the measured hydrogen concentrations, such as measured hydrogen concentrations for a specific time period, measured hydrogen concentrations over the entire deployment period, standard deviation of mean values of the measured hydrogen concentrations at specific dates and times. The interface 900 may also allow a user to select data analytics 926 for a different detected gaseous species, such as methane (CH₄), helium (He), carbon monoxide (CO), carbon dioxide (CO₂), ammonia (NH₃), and/or nitrogen dioxide (NO₂), for example via a drop-down menu (not shown).

### INTERPRETATION

This specification, including the claims, is intended to be interpreted as follows:

Embodiments or examples described in the specification are intended to be illustrative of the invention, without limiting the scope thereof. The invention is capable of being practised with various modifications and additions as will readily occur to those skilled in the art. Accordingly, it is to be understood that the scope of the invention is not to be limited to the exact construction and operation described or illustrated, but only by the following claims.

Moreover, any feature or element described within one embodiment may be combined with any feature or element as described with respect to any other embodiment detailed within this specification, as deemed suitable and appropriate by those skilled in the art.

The mere disclosure of a method step or product element in the specification should not be construed as being essential to the invention claimed herein, except where it is either expressly stated to be so or expressly recited in a claim.

The terms in the claims have the broadest scope of meaning they would have been given by a person of ordinary skill in the art as of the relevant date.

The terms “a” and “an” mean “one or more”, unless expressly specified otherwise.

Neither the title nor the abstract of the present application is to be taken as limiting in any way as the scope of the claimed invention.

Where the preamble of a claim recites a purpose, benefit or possible use of the claimed invention, it does not limit the claimed invention to having only that purpose, benefit or possible use.

It should be noted that terms of degree such as “generally”, “substantially”, “about” and “approximately” as used herein mean a reasonable amount of deviation of the modified term such that the end result is not significantly changed. These terms of degree should be construed as including a deviation of the modified term if this deviation would not negate the meaning of the term it modifies.

In the specification, including the claims, the term “comprise”, and variants of that term such as “comprises” or “comprising”, are used to mean “including but not limited to”, unless expressly specified otherwise, or unless in the context or usage an exclusive interpretation of the term is required.

Furthermore, the recitation of any numerical ranges by endpoints herein includes all numbers and fractions subsumed within that range (e.g. 1 to 5 includes 1, 1.5, 2, 2.75, 3, 3.90, 4, and 5). It is also to be understood that all numbers and fractions thereof are presumed to be modified by the term “about” which means a variation up to a certain amount of the number to which reference is being made if the end result is not significantly changed.

As used herein, the wording “and/or” is intended to represent an inclusive-or. That is, “X and/or Y” is intended to mean X or Y or both, for example. As a further example, “X, Y, and/or Z” is intended to mean X or Y or Z or any combination thereof.

The disclosure of any document referred to herein is incorporated by reference into this patent application as part of the present disclosure, but only for purposes of written description and enablement and should in no way be used to limit, define, or otherwise construe any term of the present application where the present application, without such incorporation by reference, would not have failed to provide an ascertainable meaning. Any incorporation by reference does not, in and of itself, constitute any endorsement or ratification of any statement, opinion or argument contained in any incorporated document.

1. A gas sensing apparatus configured to detect hydrogen (H₂) in the presence of one or more other gases, the apparatus including
a first sensor for detecting at least hydrogen (H₂),
a second sensor for detecting a gaseous species other than hydrogen (H₂), wherein the first sensor's response to the presence of hydrogen changes in the presence of the other gaseous species detected by the second sensor, and
a wireless transmitter for wirelessly transmitting data from the apparatus to a remote station.

2. The gas sensing apparatus of claim 1, wherein the second sensor is configured to detect any one or more of methane (CH₄), helium (He), carbon monoxide (CO), carbon dioxide (CO₂), ammonia (NH₃), and nitrogen dioxide (NO₂).

3-4. (canceled)

5. The gas sensing apparatus of claim 1, wherein the apparatus is operatively configured to calibrate data from the first and second sensors to determine a concentration of any one or more of hydrogen (H₂), methane (CH₄), helium (He), carbon monoxide (CO), carbon dioxide (CO₂), ammonia (NH₃), and nitrogen dioxide (NO₂).

6. (canceled)

7. The gas sensing apparatus of claim 1, wherein the second sensor is configured to detect at least methane (CH₄), the apparatus further including
a third sensor configured to detect any one or more of carbon monoxide (CO), carbon dioxide (CO₂), ammonia (NH₃), nitrogen dioxide (NO₂), and helium (He), wherein
the first sensor's response to the presence of hydrogen changes in the presence of any one or more of methane (CH₄), carbon monoxide (CO), carbon dioxide (CO₂), ammonia (NH₃), nitrogen dioxide (NO₂), and helium (He).

8. (canceled)

9. The gas sensing apparatus of claim 7, wherein the apparatus is operatively configured to calibrate data from the first, second and third sensors to determine a concentration of at least one of hydrogen (H₂), methane (CH₄), carbon monoxide (CO), carbon dioxide (CO₂), ammonia (NH₃), nitrogen dioxide (NO₂), and helium (He).

10-11. (canceled)

12. The gas sensing apparatus of claim 1, further including an environmental sensor configured to detect any one or more of temperature, humidity and pressure.

13. The gas sensing apparatus of claim 5, wherein the apparatus is operatively configured to perform the calibration based on one or more statistical and/or machine learning models, the one or more statistical and/or machine learning models being pre-trained on
sensor data indicative of concentrations of hydrogen (H₂) and one or more gaseous species other than hydrogen (H₂), and
actual gas concentrations of hydrogen (H₂) and one or more gaseous species other than hydrogen (H₂) corresponding to the sensor data.

14. (canceled)

15. The gas sensing apparatus of claim 13, wherein the one or more statistical and/or machine learning models includes any one of a linear regression model, gradient boosting machine learning, or random forest algorithm.

16. The gas sensing apparatus according to claim 1, further including a battery module or wherein the gas sensing apparatus is configured to receive power from one or more solar modules.

17. The gas sensing apparatus of claim 1, wherein the apparatus includes a housing for containing the sensors therein, the housing having a body, the sensors being mounted proximate a first end of the body.

18. The gas sensing apparatus of claim 17, further including a gas-permeable membrane disposed at the first end of the body to allow gases to enter the housing for detection by the sensors.

19-20. (canceled)

21. The gas sensing apparatus according to claim 18, wherein the sensors are spaced from the barrier gas-permeable membrane so as to define a cavity therebetween.

22. The gas sensing apparatus of claim 21, further including a pump or fan for periodically extracting gases from the cavity.

23-25. (canceled)

26. A gas sensing apparatus according to claim 1, wherein the sensing apparatus is a soil gas sensing apparatus, and the apparatus is configured for long term placement in the soil.

27. A system including the gas sensing apparatus of claim 1, the system further including the remote station for receiving data from the gas sensing apparatus, wherein the remote station is operatively configured to calibrate data received from the gas sensing apparatus to determine a concentration of at least one of hydrogen (H₂), methane (CH₄), carbon monoxide (CO), carbon dioxide (CO₂), ammonia (NH₃), and nitrogen dioxide (NO₂).

28-29. (canceled)

30. The system of claim 27, wherein the remote station is operatively configured to perform the calibration based on one or more statistical and/or machine learning models, the one or more statistical and/or machine learning models being pre-trained based on
sensor data indicative of concentrations of hydrogen (H₂) and one or more gaseous species other than hydrogen (H₂), and
actual gas concentrations of hydrogen (H₂) and one or more gaseous species other than hydrogen (H₂) corresponding to the sensor data.

31-42. (canceled)

43. A computer-implemented method of determining a concentration of hydrogen (H₂) in the presence of one or more other gases, the method including
receiving sensor data representing a sensed concentration of hydrogen (H₂) and a sensed concentration of a gaseous species other than hydrogen (H₂), wherein the sensed concentration of hydrogen (H₂) is influenced by the sensed concentration of the other gaseous species, and
calibrating the sensor data to determine a concentration of hydrogen (H₂).

44. The computer-implemented method of claim 43, wherein the sensor data represents a sensed concentration of hydrogen (H₂) and a sensed concentration of any one or more of methane (CH₄), helium (He), carbon monoxide (CO), carbon dioxide (CO₂), ammonia (NH₃), and nitrogen dioxide (NO₂), the method further including
calibrating the sensor data to determine a concentration of any one or more of methane (CH₄), helium (He), carbon monoxide (CO), carbon dioxide (CO₂), ammonia (NH₃), and nitrogen dioxide (NO₂).

45. (canceled)

46. The computer-implemented method of claim 43, wherein calibrating the sensor data includes calibrating the sensor data based on one or more statistical and/or machine learning models, wherein the one or more statistical and/or machine learning models is pre-trained based on
sensor data indicative of concentrations of hydrogen (H₂) and one or more gaseous species other than hydrogen (H₂), and
actual gas concentrations of hydrogen (H₂) and one or more gaseous species other than hydrogen (H₂) corresponding to the sensor data.

47-48. (canceled)

49. A non-transitory computer readable medium having stored thereon software instructions that when executed by a processor, causes the processor to perform a method according to claim 43.

50-51. (canceled)
